SEQUENCE LISTING (10 sequences)

SEQ ID NO: 1 - DNA, 21 bp, Artificial Sequence
  Forward primer for C/EBPalpha
caagaacagcaacgagtaccg

SEQ ID NO: 2 - DNA, 21 bp, Artificial Sequence
  Reverse primer for C/EBPalpha
gtcactggtcaactccagcac

SEQ ID NO: 3 - DNA, 20 bp, Artificial Sequence
  Forward primer for PPARgamma
tcgctgatgcactgcctatg

SEQ ID NO: 4 - DNA, 21 bp, Artificial Sequence
  Reverse primer for PPARgamma
gagaggtccacagagctgatt

SEQ ID NO: 5 - DNA, 21 bp, Artificial Sequence
  Forward primer for FAS
ggaggtggtgatagccggtat

SEQ ID NO: 6 - DNA, 21 bp, Artificial Sequence
  Reverse primer for FAS
tgggtaatccatagagcccag

SEQ ID NO: 7 - DNA, 15 bp, Artificial Sequence
  Forward primer for aP2
ccgcagacgacagga

SEQ ID NO: 8 - DNA, 20 bp, Artificial Sequence
  Reverse primer for aP2
ctcatgccctttcataaact

SEQ ID NO: 9 - DNA, 20 bp, Artificial Sequence
  Forward primer for beta-Actin
gcaggagtacgatgagtccg

SEQ ID NO: 10 - DNA, 20 bp, Artificial Sequence
  Reverse primer for beta-Actin
acgcagctcagtaacagtcc

(19) United States
(12) Patent Application Publication
(10) Pub. No.: US 2024/0058392 A1
(43) Pub. Date: Feb. 22, 2024

(54) COMPOSITION COMPRISING SEA CUCUMBER GENITAL GLAND EXTRACT, COMPOUND DERIVED FROM SEA CUCUMBER OVARY EXTRACT, AND USE THEREOF

(71) Applicants: BIOINFORMATICS & MOLECULAR DESIGN RESEARCH CENTER, Incheon (KR); BENTHIC BIO INC., Incheon (KR)

(72) Inventors: Kyoung Tai NO, Incheon (KR); Suk-Kyu CHANG, Gyeonggi-do (KR); Ji Young LEE, Incheon (KR); Ara JANG, Gyeonggi-do (KR); Zuhkyung SEONG, Gyeonggi-do (KR); Jin Hwan NO, Chungcheongnam-do (KR); Jiwon LEE, Incheon (KR); Sohee AN, Incheon (KR)

(21) Appl. No.: 18/266,396

(22) PCT Filed: Dec. 9, 2021
(86) PCT No.: PCT/KR2021/018696

§ 371 Date: Jun. 9, 2023

(30) Foreign Application Priority Data
Dec. 10, 2020 (KR) 10-2020-0172701

(51) Int. Cl. A61K 35/616; A61P 3/04; A61P 3/10

(52) U.S. Cl. CPC A61K 35/616; A61P 3/04; A61P 3/10

(57) ABSTRACT

The present invention provides a sea cucumber genital gland extract, preferably, a novel saponin compound separated from an ovary extract, and a novel use of a sea cucumber genital gland extract or a compound separated from a sea cucumber genital gland extract. The present invention has an effect of treating or preventing any one or more diseases selected from the group consisting of obesity, inflammation, diabetes and dyslipidemia, by comprising a sea cucumber genital gland, preferably, an ovary extract, or a compound separated from a sea cucumber genital gland, preferably, an ovary extract.

### TECHNICAL FIELD

The present application claims the priority based on Korean Patent Application No. 10-2020-0172701 filed on Dec. 10, 2020, and the entire contents disclosed in the description and drawings of the corresponding application are incorporated in the present application. The present invention relates to a novel use of a sea cucumber genital gland extract, preferably, a use for anti-obesity, anti-diabetes, improvement of dyslipidemia, and/or improvement of inflammatory disease of a sea cucumber genital gland extract, and relates to a new compound separated from a sea cucumber genital gland extract, or a use of the compound, preferably, a use for anti-obesity, anti-diabetes, improvement of dyslipidemia, and/or improvement of inflammatory disease.

### BACKGROUND

Sea cucumber (scientific name, Stichopus japonicus) is a traditional seafood used as an important food ingredient in Asian countries, particularly, China, Japanese and Korea. There are many biologically active substances separated from sea cucumbers. In particular, collagen peptides, polysaccharides, and the like are well known to exhibit various biological activities. An ovary of a sea cucumber is called Haeseoja and is famous as a delicacy.

On the other hand, in adipocytes, lipid metabolism occurs through lipolysis and adipogenesis, and during fat synthesis, the preadipocytes are differentiated to mature adipocytes through proliferation and differentiation processes, and ultimately, fat accumulates in the cells. As transcription factors inducing the process of differentiation of the adipocytes, PPARγ (peroxisome proliferator activated receptor γ) and C/EBPα (CCAAT enhancer binding protein α), and the like have been reported. The transcription factors induce expression at each different time point during the process of differentiation of adipocytes, regulates expression of genes specific adipocytes through interaction with each other, and gradually induce activation of fat metabolism and differentiation of adipocytes. They are known to be involved in lipogenesis and adipogenesis, which induce obesity.

Dyslipidemia is a disease name including hyperlipidemia, hypercholesterolemia, and hypertriglyceridemia. This is a state in which total cholesterol, LDL cholesterol, and triglyceride are increased or a state in which HDL cholesterol is reduced, in blood. Dyslipidemia is diagnosed when any one of those abnormalities is found in the criteria of total cholesterol 200 mg/dL or less, LDL cholesterol 130 mg/dL or less, HDL cholesterol 60 mg/dL or more, and triglyceride 150 mg/dL or less through a blood test. Diagnosis of dyslipidemia is diagnosing through lipid indicators such as total cholesterol, low density lipoprotein (LDL) cholesterol, high density lipoprotein (HDL) cholesterol, triglyceride, and the like according to U.S. NCEP (National Cholesterol Education Program) accepted universally.

Type 2 diabetes is a metabolic disease characterized by insulin resistance and dysfunction of pancreatic beta cells and a blood sugar increase thereby, and occurrence of the disease shows a complex genetic tendency. Several research results that polymorphism of many genes is involved in occurrence of type 2 diabetes have been reported. In particular, PPARγ (Peroxisome proliferator-activated receptor-gamma) gene is a gene considered as the relationship with insulin resistance is important. The PPARγ is specific to adipocytes, and is involved in differentiation of adipocytes and fatty acid metabolism, and a therapeutic agent for diabetes, thiazolidinediones is known as a ligand, so it has been pointed out that it may be a causative gene related to type 2 diabetes and obesity.

In particular, a lipid activation transcription factor, PPARγ has various functions in an immune system, and it has been known that inflammation and anti-inflammation responses can be regulated through inhibition of this transcription factor, and it has been known that it is involved in treatment of inflammatory bowel disease, or rheumatoid arthritis through this (PPARγ in immunity and inflammation: cell types and diseases, Biochimica et Biophysica Acta 1771 (2007) 1014-1030).

However, reports on a substance which has an excellent effect of anti-obesity, prevention or treatment of diabetes or dyslipidemia, or improvement or treatment of inflammation and is safe are still insufficient.

### DISCLOSURE

### Technical Problem

Accordingly, a problem to be solved by the present invention is to provide a new compound, particularly, a saponin compound, separated from a sea cucumber. A problem to be solved by the present invention is to provide a novel use for prevention or treatment of diseases such as anti-obesity, anti-diabetes, improvement of dyslipidemia, and/or improvement of inflammatory disease of a sea cucumber genital gland extract, preferably, an ovary extract, and a saponin compound derived from the extract. In addition, a composition for treatment or prevention of obesity, diabetes, or inflammatory disease comprising a sea cucumber genital gland extract, or a saponin compound derived from the extract is to be provided.

### Technical Solution

In order to solve the above problems, the present invention provides a novel compound represented by the following chemical formula S1, S2, S3, S4 and/or S9.

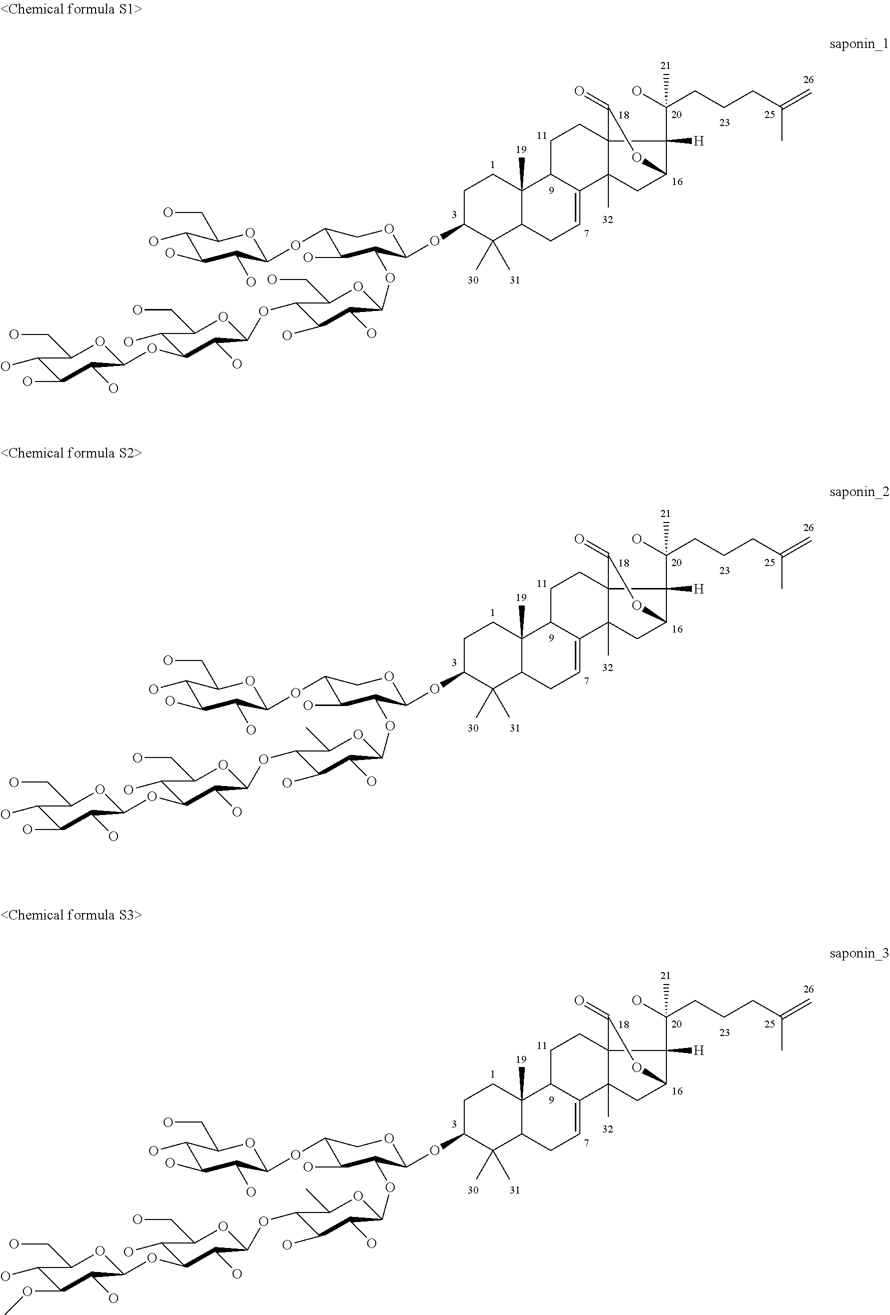

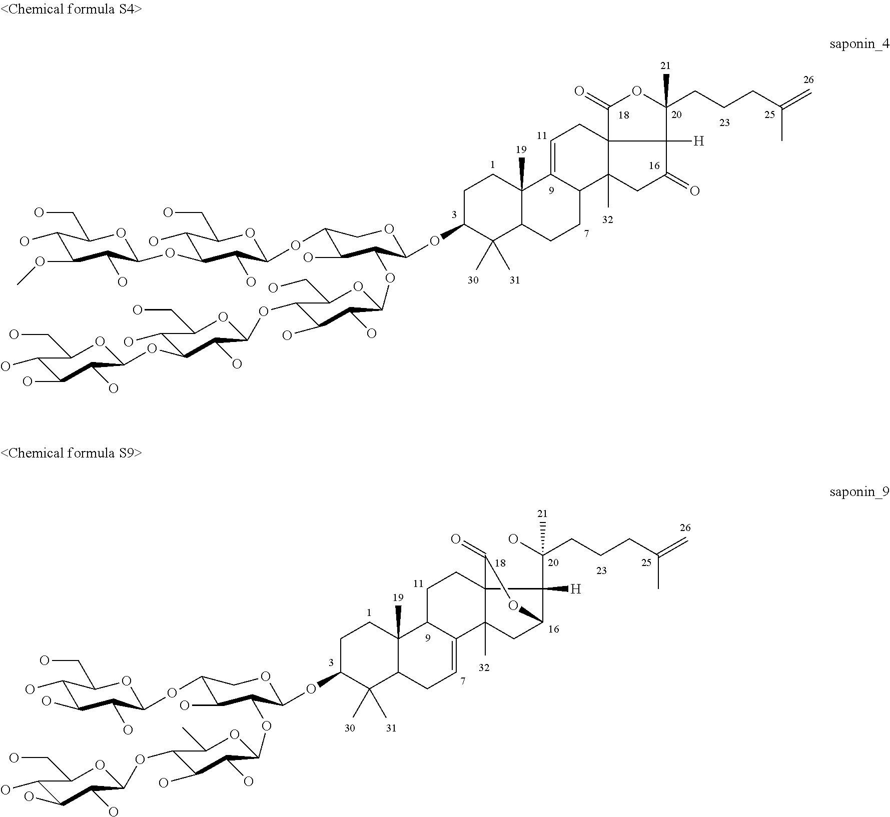

The compound may be derived from nature, and may be synthesized by a chemical synthesis method, and a process of obtaining the compound is not particularly limited. In one example of the present invention, the compound may be extracted or separated from a sea cucumber genital gland, and preferably, it may be extracted or separated from a sea cucumber ovary.

Another example of the present invention provides a composition for prevention or treatment of any one disease selected from the group consisting of obesity, inflammation, diabetes and dyslipidemia, comprising a sea cucumber genital gland extract, or a saponin compound derived from a sea cucumber genital gland. Preferably, the diabetes may be type 2 diabetes.

The sea cucumber ovary extract means that a sea cucumber genital organ, an ovary is extracted with a solvent. It is known that as a sea cucumber genital gland matures, a testicle turns milky white and an ovary turns red. The present invention may use an extract of a sea cucumber genital gland, preferably, an ovary. As the solvent, water, C1˜C4 lower alcohol, or a mixed solvent thereof may be used, and preferably, it is preferable to use ethanol or an ethanol aqueous solution. When the solvent is used, in particular, an active ingredient extraction yield is excellent and it is advantageous in achieving the object of the present invention. The extract of the present invention may be prepared according to a preparation method of an extract using a common animal as a subject of extraction, and specifically, it may be an enfleurage extraction method, a maceration extraction method or a heat extraction method, or the like, and it may use a common extraction device, a sonication extractor or a fractionator. Preferably, the sea cucumber genital gland extract, preferably, ovary extract may be extracted with a 45 to 85% (V/V) ethanol aqueous solution as a solvent, and more preferably, the sea cucumber ovary may be extracted with a 48 to 55% (V/V) ethanol aqueous solution as a solvent, or a 75 to 83% (V/V) ethanol aqueous solution as a solvent. When the solvent is used, it may be advantageous in achieving the object of the present invention.

In other example, the sea cucumber genital gland, preferably, ovary may go through a process of fractionizing an extract, and the sea cucumber ovary fraction obtained therefrom may be included in the scope of the present invention. Preferably, an extract that a sea cucumber ovary is extracted with an ethanol aqueous solution may be fractionized, and preferably, the ethanol aqueous solution may be a 45 to 85% (V/V) ethanol aqueous solution. The fraction may be preferably, a sea cucumber ovary extract fractionized with butanol as a solvent. The fraction may be obtained by hexane, methylene chloride, ethyl acetate, butanol, water or a combination thereof using a separatory funnel after suspending the sea cucumber ovary extract in distilled water and the like, and preferably, on the purpose of the present invention, the sea cucumber ovary extract may be fractionized with butanol. The fractionization may be performed by a fractionization method commonly used in the art.

In the prepared extract or fraction obtained by performing the fractionization process, a solvent may be removed by performing a subsequent process of filtration or concentration or drying, and filtration, concentration and drying may be all performed. Specifically, the filtration may use filter paper, or use a decompression filter, and the concentration may conduct decompression concentration using a decompression concentrator, a rotary evaporator as one example, and the drying may be performed by a lyophilization method as one example.

The extract or fraction may have an excellent effect of reducing body weight, and may have inhibitory activity of adipocyte differentiation, and may have an excellent effect of improving inflammation, and may have an excellent effect of reducing cholesterol, and may have an effect of treating or improving diabetes.

In one example of the present invention, the sea cucumber genital gland, preferably, ovary extract includes any one or more selected from the group consisting of the following chemical formulas S1, S2, S3, S4, S5, S6, S8 and S9, preferably, any one or more saponin compounds selected from S3, S4, S5, S6 and S8. Preferably, the following compounds separated from the sea cucumber ovary extract may have an effect of treatment or prevention of any one or more diseases selected from the group consisting of obesity, inflammation, diabetes and dyslipidemia.

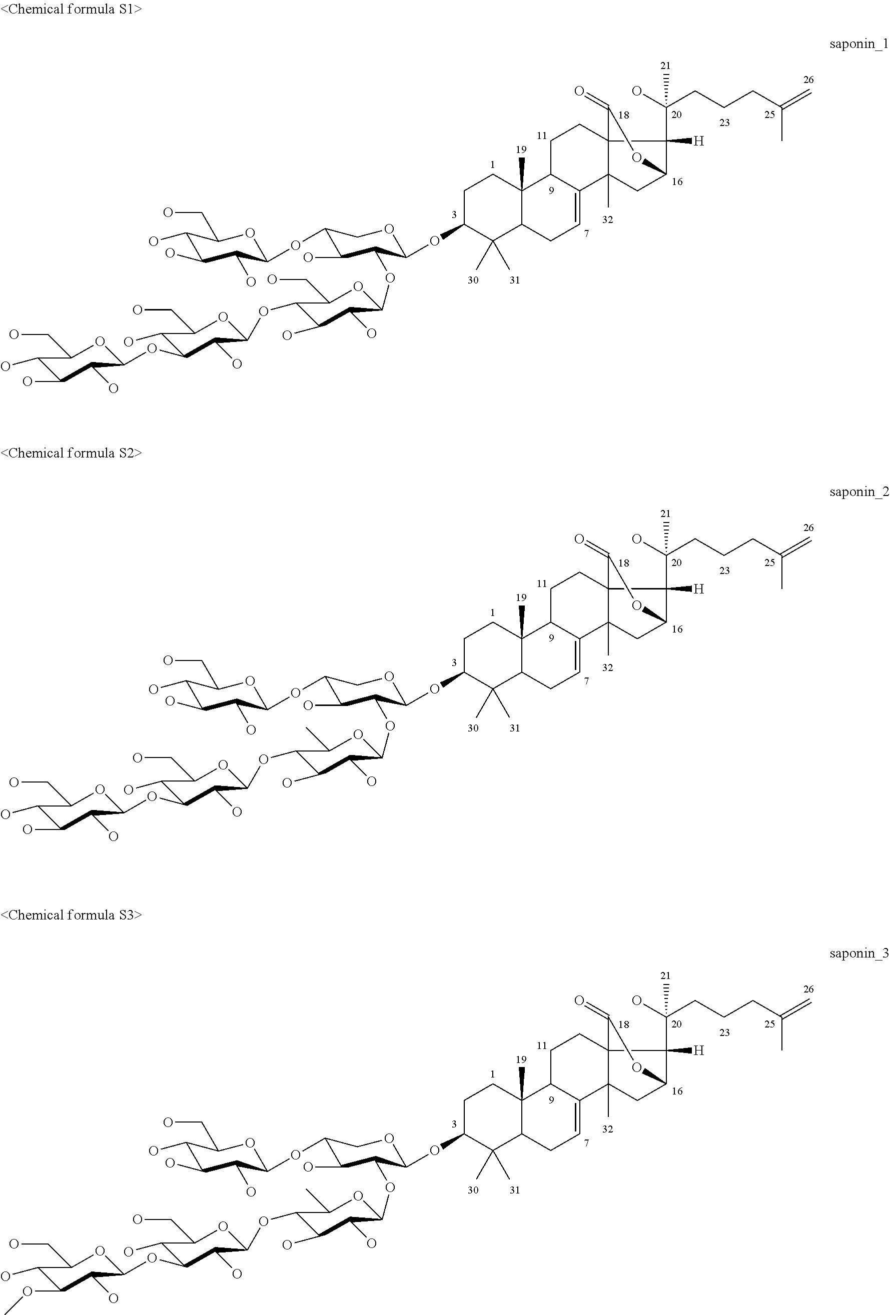

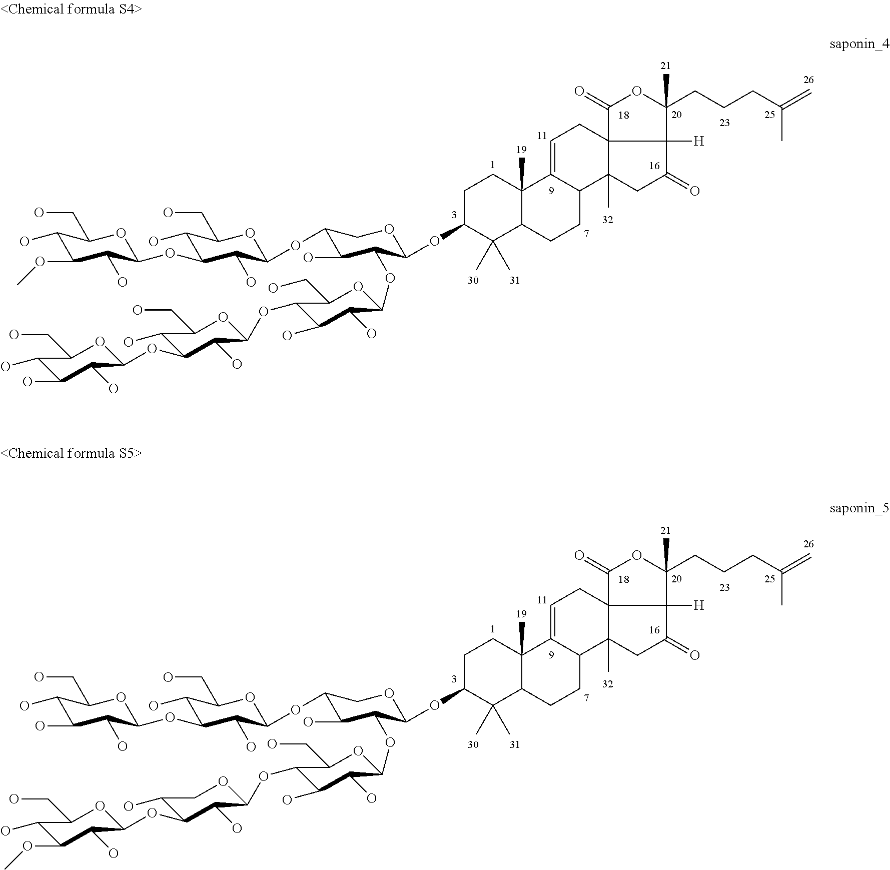

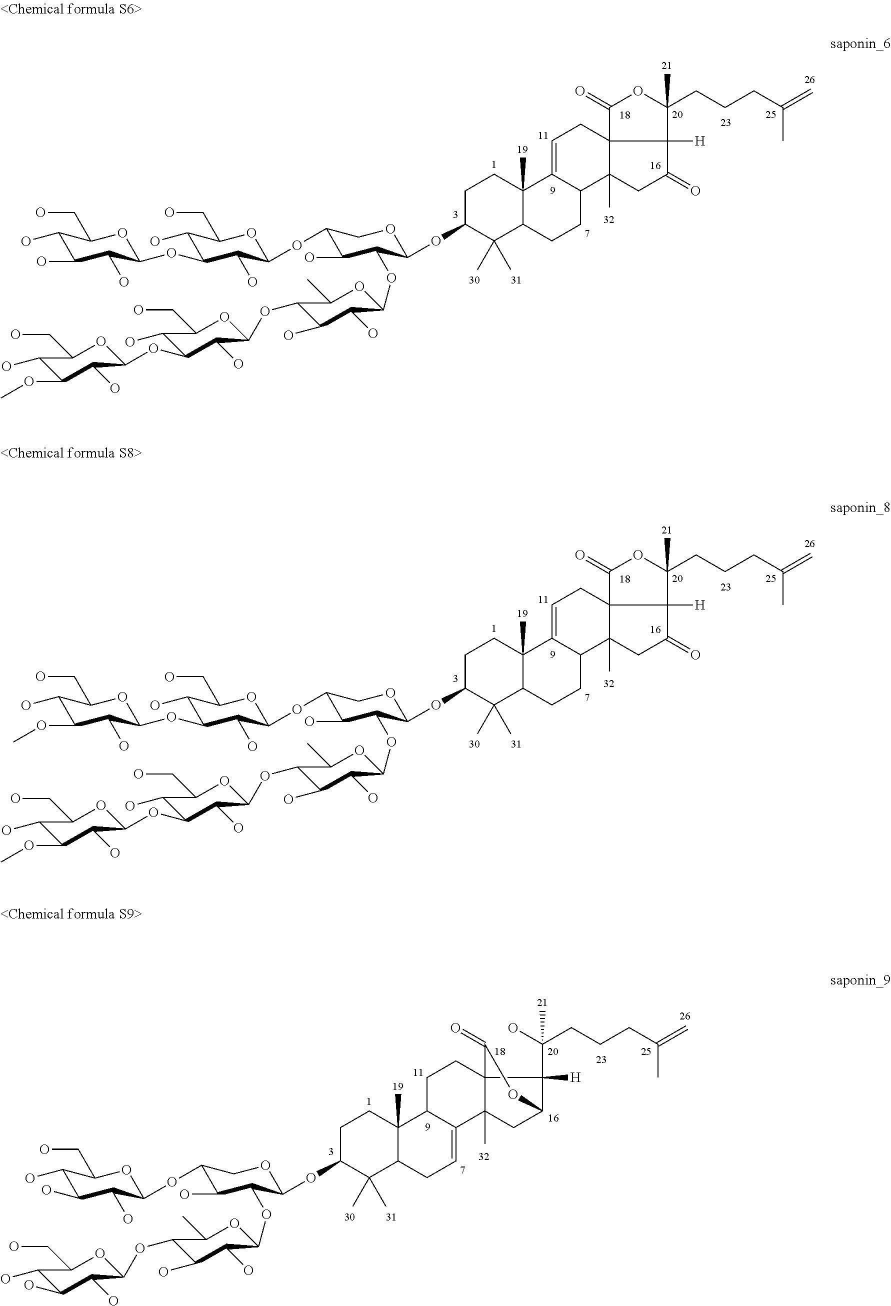

The sea cucumber genital gland, preferably, ovary extract, or saponin compound derived from a sea cucumber ovary may inhibit expression of any one or more proteins selected from the group consisting of PPARγ (Peroxisome proliferation activated receptor-γ), FAS (FS-7-associated surface antigen), aP2 (Adipocyte protein 2), and C/EBPα (CCAAT/Enhancer-binding Protein α).

The sea cucumber genital gland, preferably, ovary extract, or saponin compound derived from a sea cucumber ovary may inhibit differentiation from preadipocytes to adipocytes, or inhibit intracellular fat accumulation. Otherwise, it may have an effect of inhibiting diabetes.

The sea cucumber genital gland, preferably, ovary extract, or saponin compound derived from a sea cucumber ovary may be used as a use for weight loss, reduction of abnormal lipids in blood, relief of lipid metabolism abnormality of a subject, a use for inhibiting or improving inflammation, or a use for treating or improving diabetes.

One example of the present invention can provide a method for inhibiting, improving or alleviating fat accumulation in a body, or reducing body weight or reducing abnormal lipids in blood, or inhibiting body fat accumulation of a subject, a method for improvement or inhibition of inflammation, or a method for treating or improving diabetes, by treating an extract of a sea cucumber genital gland, preferably, ovary, or a saponin compound derived from a sea cucumber ovary.

One example of the present invention can provide a method for preparing a composition for inhibiting body fat accumulation of a subject, a composition for treating or improving dyslipidemia of a subject, a composition for treatment or improvement of inflammation-related diseases of a subject, or a composition for treating or improving diabetes comprising the following steps.
S1) separating a genital gland, preferably, ovary from a sea cucumber;
S2) treating the genital gland of the sea cucumber separated in the step S1) with a solvent, preferably, an ethanol solvent, more preferably, a 45 to 85% (V/V) ethanol aqueous solution solvent;
S3) stirring at a room temperature for 24 hours, after treating the genital gland of the sea cucumber with the solvent; and
S4) obtaining a sea cucumber extract by decompression concentrating a solution filtered after filtering.

One embodiment of the present invention can provide a food or medicine for improving or treating obesity, a food or medicine for improving or treating diabetes (particularly, type 2 diabetes), a food or medicine for improving or treating dyslipidemia (for example, hyperlipidemia, hypercholesterolemia, hypertriglyceridemia), or a food or medicine for improving or treating inflammation (for example, inflammatory bowel disease, rheumatoid arthritis), which comprises the sea cucumber genital gland extract disclosed herein.

The food or medicine may comprise an excipient, an additive, and the like commonly used in the art without limitation, within a range without inhibiting the purpose of the present invention.

One embodiment of the present invention can provide a method for inhibiting expression of any one or more proteins selected from the group consisting of PPARγ, FAS, aP2, and C/EBPα of a subject in need thereof by treating a sea cucumber ovary extract.

The composition according to one example of the present invention can be used in any field to which a novel use for treatment, prevention and/or improvement and the like of any one or more diseases selected from the group consisting of obesity, inflammation, diabetes, and dyslipidemia of the sea cucumber genital gland, preferably, ovary extract, or saponin compound derived from a sea cucumber genital gland, preferably, ovary of the present invention can be applied without limitation, and preferably, it may be provided in a pharmaceutical composition, or food composition form. Preferably, the food composition may be provided as a health functional food composition.

When the composition of the present invention is prepared as a pharmaceutical composition, the pharmaceutical composition of the present invention comprises a pharmaceutically acceptable carrier. The pharmaceutically acceptable carrier comprised in the pharmaceutical composition of the present invention is one commonly used during formulation, and includes lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, methyl cellulose, methylhydroxybenzoate, talc, magnesium stearate, and mineral oil and the like, but not limited thereto. The pharmaceutical composition of the present invention may further comprise a lubricant, a wetting agent, a sweetener, a flavoring agent, an emulsifier, a suspending agent, a preservative, and the like in addition to the above components. The pharmaceutical carrier is a non-restrictive example, and is not limited to the above kinds. The pharmaceutical composition of the present invention may be orally or parenterally administered, and preferably, it may be applied by an oral administration method. An appropriate dosage of the pharmaceutical composition of the present invention may be variously prescribed by factors such as formulation method, administration method, patient's age, body weight, gender, morbid condition, food, administration time, administration route, excretion rate and reaction sensitivity. A preferable dosage of the pharmaceutical composition may be within a range of 0.001-100 mg/kg on an adult basis. The pharmaceutical composition of the present invention may be prepared in a form of a unit dose or be prepared by inserting in a multi-dose container, by formulating using a pharmaceutically acceptable carrier and/or excipient, according to a method which can be easily conducted by those skilled in the art to which the present invention pertains. Then, the formulation may be in the form of a solution, suspension, syrup or emulsion in an oil or aqueous medium, or may be in the form of an extract, powder, dry powder, granule, tablet or capsule, and may further comprise a dispersing agent or stabilizer.

When the composition of the present invention is prepared as a food composition, it comprises a commonly added component during preparation of foods, and for example, it comprises a protein, a carbohydrate, a fat, a nutrient, a seasoning and a flavoring agent. The example of the carbohydrate described above is a monosaccharide, for example, glucose, fructose, etc.; a disaccharide, for example, maltose, sucrose, oligosaccharide, etc.; and a polysaccharide, for example, a common sugar such as dextrin, cyclodextrin and the like and a sugar-alcohol such as xylitol, sorbitol, erythritol, and the like. As the flavoring agent, a natural flavoring agent such as stevia extract, or a synthetic flavoring agent such as saccharin, and the like may be used.

One example of the present invention provides a method for improving an effect of prevention or treatment of any one or more diseases selected from the group consisting of obesity, inflammation, diabetes, and dyslipidemia of a sea cucumber genital gland, preferably, ovary extract, by increasing any one or more saponin compounds selected from the group consisting of the chemical formulas S1, S2, S3, S4, S5, S6, S8 and S9, preferably, any one or more saponin compounds selected from the group consisting of S3, S4, S5, S6 and S8. All the methods for obtaining the saponin compounds mentioned in the present invention may be used, and preferably, as the method, a method for extracting a sea cucumber genital gland, preferably, ovary with an ethanol aqueous solution, and obtaining a butanol fraction from the extract may be used, and preferably, a method for extracting it with a 45 to 85% (V/V) ethanol aqueous solution, and conducting butanol fractionization from the extract may be used.

One embodiment of the present invention provides a method for treating or improving any one disease selected from the group consisting of obesity, inflammation, diabetes and dyslipidemia, comprising administering a therapeutically effective amount of a sea cucumber genital gland extract or a saponin compound derived from a sea cucumber genital gland into a subject in need thereof. It may be understood that the saponin compound derived from the sea cucumber genital gland mentioned herein comprises any one or more selected from the group consisting of the chemical formulas S1, S2, S3, S4, S5, S6, S8 and S9. Other embodiment may provide a method for treating or improving any one disease selected from the group consisting of obesity, inflammation, diabetes and dyslipidemia, which increases a sea cucumber genital gland extract or a saponin compound derived from a sea cucumber genital gland in a subject in need thereof. Other embodiment may provide a composition for treatment or improvement of any one disease selected from the group consisting of obesity, inflammation, diabetes and dyslipidemia, comprising a sea cucumber genital gland extract, or a saponin compound derived from a sea cucumber genital gland, and a pharmaceutically acceptable carrier. Other embodiment may provide a use for treating or improving any one disease selected from the group consisting of obesity, inflammation, diabetes and dyslipidemia of a sea cucumber genital gland extract, or a saponin compound derived from a sea cucumber genital gland. Other embodiment may provide a method for inhibiting expression of any one or more proteins selected from the group consisting of PPARγ, FAS, aP2, and C/EBPα in a body, comprising administering a therapeutically effective amount of a sea cucumber genital gland extract, or a saponin compound derived from a sea cucumber genital gland into a subject in need thereof.

### Advantageous Effects

The present invention provides a novel saponin compound unknown conventionally.

The present invention suggests a new use of a sea cucumber genital gland, preferably, an ovary extract, or a saponin compound separated therefrom. Through the present invention, an anti-obesity effect, an effect of reducing body weight, an effect of treating or improving diabetes, an anti-inflammation effect, an effect of preventing, improving or treating dyslipidemia, and the like of a sea cucumber genital gland, preferably, an ovary extract, excellent compared to other parts of the sea cucumber have been confirmed. Through the present invention, an effect of prevention, improvement or treatment of any one or more diseases selected from the group consisting of obesity, inflammation, diabetes and dyslipidemia can be obtained.

### BRIEF DESCRIPTION OF THE DRAWINGS

The following drawings attached in the present description illustrate preferable examples of the present invention, and play a role of understanding the technical spirit of the present invention with the contents of the invention described above, so the present invention should not be interpreted as limited only to the matters described in such drawings.

FIG. 1 is the result of confirming the cell viability of cells after treating an extract obtained from a sea cucumber ovary extract and internal organs except for an ovary by varying ethanol concentrations to 3T3-L1 cells.

FIG. 2a is a drawing which shows an effect of inhibiting differentiation of adipocytes and fat accumulation of a sea cucumber extract.

FIG. 2b shows O.D. values confirmed after treating Samples 1-4.

FIG. 3 shows the result of analyzing adipocyte differentiation-related genes and proteins by part and concentration of a sea cucumber extract. FIG. 3a is the result of showing the degree of protein expression, and the graphs of FIG. 3b are the result of confirming the effect on mRNA expression of a sea cucumber extract.

FIG. 4a is photographs showing adipocytes differentiation of a sea cucumber extract,

and FIG. 4b is a graph showing the effect of inhibiting fat accumulation (25, 50 μg/ml).

FIG. 5 is the result of confirming the effect on expression of proteins involved in a process of adipocyte differentiation, PPARγ, FAS, aP2, and C/EBPα of a sea cucumber extract. It was confirmed that the sea cucumber ovary extract inhibits these proteins.

FIG. 6 shows a process of separating a compound through extraction and fractionization of a sea cucumber ovary extract as a diagram.

FIG. 7 shows an effect on the 3T3-L1 cell viability of compounds separated from a sea cucumber ovary extract.

FIG. 8a shows the result of analyzing the degree of fat accumulation by compounds separated from a sea cucumber ovary extract through Oil red O staining after completing adipose differentiation of 3T3-L1 cells.

FIG. 8b shows differentiated 3T3-L1 cells, and

FIG. 8c shows optical density (O.D.) of Oil Red O measured at 500 nm. They were treated at a concentration of 2.5, 5 μg/ml, respectively.

Data are presented mean±standard deviation (SD). *, **, and *** indicate p<0.05, 0.01, and 0.001, respectively, when compared to the differentiated control group (MDI).

FIG. 9 shows an adipogenesis-related protein level of differentiated 3T3-L1 cells when S1-S6, S8-S9 compounds are treated, respectively.

FIG. 10 is the result of confirming the glucose uptake of a sea cucumber ovary extract and an extract of internal organs except for an ovary of a sea cucumber.

FIG. 11 is the result of confirming the glucose uptake by condition of extraction of a sea cucumber ovary extract.

FIG. 12 is the result of confirming the glucose uptake of saponin compounds separated from an ovary extract of a sea cucumber.

FIG. 13 is the result of observing a change in body weight.

FIG. 14 shows the final body weight confirmed after treating an extract of a high calorie diet intake.

FIG. 15 is the result of white adipocyte size change confirmed through H&E stain.

FIG. 16 is the result of representing an HDL content compared to total cholesterol.

FIG. 17 is the result of confirming an effect of reducing total cholesterol.

FIG. 18 shows the result of confirming an effect of reducing triglyceride.

FIG. 19 is the result of confirming an effect of reducing PPARγ expression of an extract treatment group.

### MODE FOR INVENTION

Hereinafter, in order to help understanding of the present invention, it will be described in detail by examples and the like. However, the examples according to the present invention may be modified into various other forms, and the scope of the present invention should not be interpreted as limited by the following examples. The examples of the present invention are provided to more completely describe the present invention to those skilled in the art to which the present invention pertains.

### I. Experimental Example 1—Confirmation of Effect of Extract by Sea Cucumber Part

1. Sample Extraction

(1) Extraction Part and Solvent Concentration

TABLE 1

| | Part | Solvent | |
|---|---|---|---|
| Sea cucumber | Intestinal | 1 | 50% EtOH |
| (Stichopus japonicus) | organ | 2 | 80% EtOH |
| | Ovary | 3 | 50% EtOH |
| | | 4 | 80% EtOH |

A sea cucumbers was divided into an ovary and intestinal organ excluding ovaries, and these were extracted. 50% ethanol and 80% ethanol were added 20 times each to the sea cucumber ovary and intestinal organs in the dried state, and they were stirred at a room temperature for 24 hours to extract. After that, through a process of filtration and decompression concentration, sea cucumber ovary and intestinal organ extracts were obtained.

2. Evaluation of Inhibitory Ability of Adipocyte Differentiation of Sea Cucumber Extract

(1) Experimental Materials and Methods

Reagents and materials are as follows.

3T3-L1 cells (ATCC, CL-173), DMEM high glucose (Gibco), fetal bovine serum (Gibco), penicillin-streptomycin-glutamin (Gibco), phosphate buffered saline (Gibco), Radioimmunoprecipitation assay (RIPA) buffer (Thermo scientific), protease- and phosphatase-inhibitor cocktails (Thermo scientific), PVDF membrane (Bio-rad), Primary antibody (PPARγ (Santa cruz), C/EBPα (cell signaling), FAS (cell signaling), aP2 (cell signaling), β-actin (Santa cruz), Oli red O powder (sigma), and MTT powder (sigma) were used.

(2) Measurement of Cell Viability

3T3-L1 cells were aliquoted in a 96 well plate at a concentration of 1×10⁴ cells/well and cultured for a day, and then 6 kinds of extracts were treated at a concentration of 100-200 μg/ml. After 24 hours, MTT powder was dissolved in PBS at a concentration of 5 mg/ml and then filtered, and 20 μl of cells was aliquoted each and cultured for 2 hours. After skimming the medium, 200 μl of DMSO was aliquoted, and then the absorbance was measured at 570 nm to measure the cell viability.

(3) Culture of 3T3-L1 Preadipocytes and Differentiation of Adipocytes

3T3-L1 cells were cultured in a high glucose DMEM medium in which 10% calf serum and penicillin streptomycin glutamin were added and subcultured per 2-3 days. After aliquoting the 3T3-L1 cells in a 96 well plate at a concentration of 4×10⁵ cells/well and then culturing them for 2 days, it was replaced with an MDI medium that dexamethasone, insulin, and 3-isobuty-1-methylxanthine (IBMX) were added to a medium containing 10% FBS instead of calf serum. After 2 days, it was replaced with a medium containing insulin, and the medium was replaced again per 2 days and differentiation was induced. The sea cucumber extract was treated at a concentration of 200 μg/ml in 30 minutes after replacing the medium.

(4) Oil Red O Staining

The 3T3-L1 cells in which differentiation was completed were fixed with 4% formaldehyde for 1 hour, and then fats were stained using a 0.5% Oil red O staining solution to confirm the degree of differentiation. For quantitative analysis of adipose differentiation, the dye stained in the cells was dissolved in 100% isopropyl alcohol, and the absorbance was measured at 500 nm.

(5) Quantitative Real-Time PCR

Primer sequences were shown in Table 2.

TABLE 2

| Gene | Forward primer (5′→3′) | Reverse primer (5′→3′') |
|---|---|---|
| C/EBPα | SEQ ID NO: 1 | SEQ ID NO: 2 |
| PPARγ | SEQ ID NO: 3 | SEQ ID NO: 4 |
| FAS | SEQ ID NO: 5 | SEQ ID NO: 6 |
| aP2 | SEQ ID NO: 7 | SEQ ID NO: 8 |
| β-Actin | SEQ ID NO: 9 | SEQ ID NO: 10 |

In order to investigate the effect on expression of adipose differentiation-related genes of the sea cucumber extract, the degree of mRNA expression of the related genes was confirmed using qRT-PCR. The differentiation-completed 3T3-L1 cells were skimmed using PBS and then it was extracted using Rneasy Mini kit (Qiagen), and RT-PCR was carried out by an SYBR green method by synthesizing cDNA (ReverTra Ace qPCR RT Master Mix, FSQ-201, TOYOBO) with 1 μg of the extracted RNA. It was proceeded with a PCR condition of 40 cycles of pre-denaturation (95° C., 1 minute), 95° C., 15 seconds, and 60° C., 15 seconds. The used primer sequences were as Table 2.

(6) Western Blotting

In order to investigate the effect on expression of adipocyte differentiation-related proteins of the sea cucumber extract, expression of the related proteins was confirmed through western blot. The differentiation-completed 3T3-L1 cells were washed with PBS, and then they were lysed with RIPA buffer, and then proteins were separated by molecular weight through SDS-PAGE. After that, they were transferred to a PVDF membrane and blocking was performed using 5% skim milk for 30 minutes. Then, after reacting with a primary antibody, a secondary antibody was attached and protein expression was confirmed using an ECL solution.

(7) Statistics

Statistical analysis of the result was performed using GraphPad Prism 7 software (San Diego, CA, USA), and a significant difference between each group was analyzed through One-way ANOVA. (p<0.05)

3. Experimental Result

(1) Cell Viability

As a result of investigating the effect on the viability of the 3T3-L1 cells of the sea cucumber extract, according to each condition, all of the 6 extracted sea cucumber extracts did not show a significant difference from a non-treatment group up to 200 μg/ml.

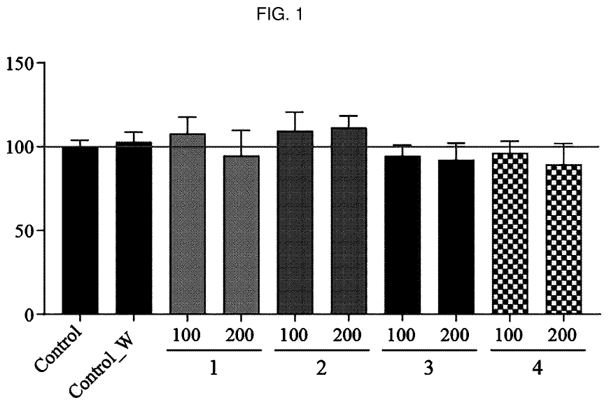

After treating the sea cucumber extract, the viability of the 3T3-L1 cells was shown in FIG. 1.

(2) Oil Red O Staining Result

In order to investigate the effect on adipose differentiation of the 3T3-L1 cells by the sea cucumber extract, as a result of analyzing the degree of fat accumulation through Oil red O staining, in the cells treated with the sea cucumber ovary 50% and 80% ethanol extracts, the degree of staining was reduced, and an effect of reducing fat accumulation was shown. In addition, in order to quantitively represent this effect, as the result of measuring the absorbance by dissolving Oil red O stained in the cells with 100% isopropyl alcohol, in the cells treated with the sea cucumber ovary 50% and 80% ethanol extracts, it was confirmed that the absorbance was significantly reduced and fat accumulation was reduced.

In other words, it was confirmed that the sea cucumber ovary extract had an effect of inhibiting fat accumulation compared to other organs of the sea cucumber.

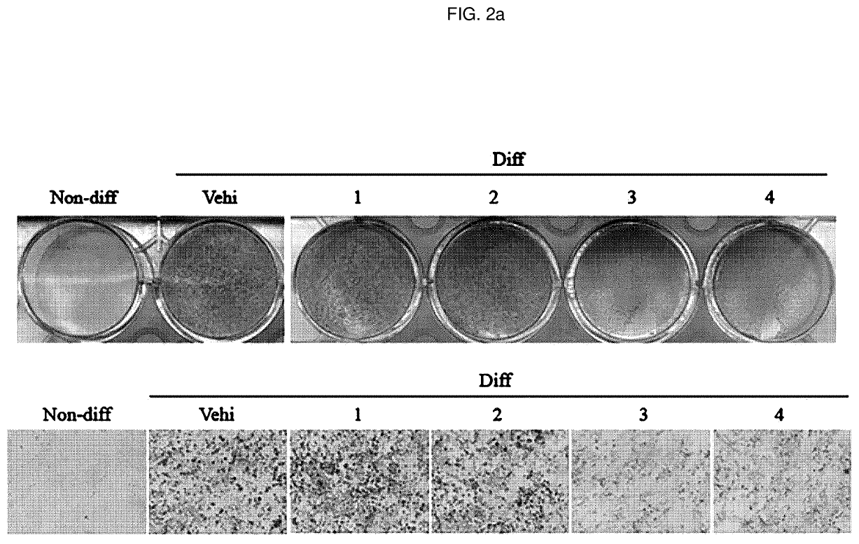

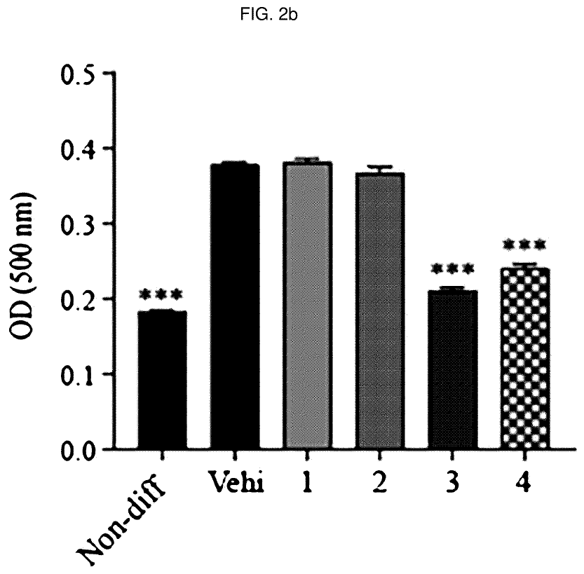

In FIG. 2, the effect of inhibiting adipocyte differentiation and fat accumulation of the sea cucumber extract was shown. As could be confirmed in FIG. 2, in the treatment groups 3 and 4 (ovary extracts), fats were less accumulated compared to the treatment groups 1 and 2, so the O.D. value was almost half of that of the treatment groups 1 and 2.

(3) Analysis of Adipocyte Differentiation-Related Genes and Proteins

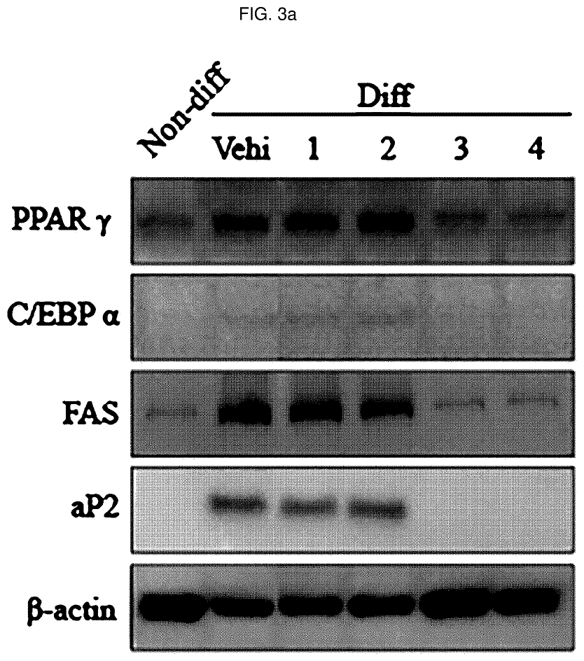

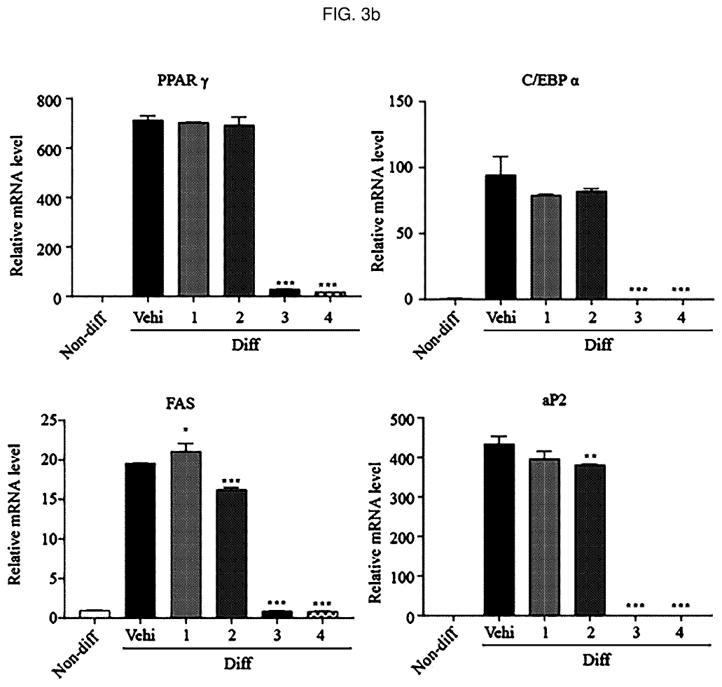

As a result of confirming the effect of the sea cucumber extract affecting expression of proteins and mRNA of PPARγ, FAS, aP2, C/EBPα known to play an important role in the adipocyte differentiation process, it could be confirmed that the protein expression was reduced in the cells treated with the sea cucumber ovary 50% and 80% ethanol extracts, similarly to the previous experiment. In addition, it could be confirmed that the mRNA expression was also significantly reduced in the cells treated with the sea cucumber ovary 50% and 80% ethanol extracts compared to other intestinal organ parts of the sea cucumber. Therefore, taking these results together, the sea cucumber extract seems to inhibit adipose differentiation by regulating expression of the genes and proteins related to adipose differentiation. In FIG. 3, the result of the effect of inhibiting expression of the adipose differentiation-related genes and proteins of the sea cucumber extract was shown.

When comparing the effect of inhibiting adipose differentiation of the sea cucumber extract extracted according to each condition, the most effective extraction condition was confirmed as the sea cucumber ovary 50% (Sample name: SJ-O-D1-50) or 80% ethanol extract (Sample name: SJ-O-D1-80). In other words, the treatment groups 3 and 4 had the excellent effect of inhibiting expression of the adipose differentiation-related genes.

### II. Experimental Example 2—Verification of Effect of Extract and Effect of Fraction According to Solvent Condition

1. Experimental Materials and Methods

(1) Preparation of Sea Cucumber Samples

TABLE 3

| | Sample name | Extract information |
|---|---|---|
| 1 | SJ-O-D1-30 | Dried ovary 30% EtOH, 20-fold, 24 h stirring, primary |
| | | extraction |
| 2 | SJ-O-D1-50 | Dried ovary 50% EtOH, 20-fold, 24 h stirring, primary |
| | | extraction |
| 3 | SJ-O-D1-80 | Dried ovary 80% EtOH, 20-fold, 24 h stirring, primary |
| | | extraction |
| 4 | SJ-O-D1-H | Hexane fraction of Sample 2 |
| 5 | SJ-O-D1-B | BuOH fraction of Sample 2 |

(2) Reagents and Materials

They were prepared as the items of I-2-(1).

(3) Culture of 3T3-L1 Preadipocytes and Differentiation of Adipocytes

The 3T3-L1 cells were cultured in a high glucose DMEM medium in which 10% calf serum and penicillin streptomycin glutamine were added, and subcultured per 2-3 days. The 3T3-L1 cells were aliquoted in a 96 well plate at a concentration of 4×10⁵ cells/well and then it was replated with an MDI medium that dexamethasone, insulin, and 3-isobuty-1-methylxanthine (IBMX) were added to a medium containing 10% FBS instead of calf serum, and the sea cucumber extract was treated in 30 minutes after replacing with the MDI medium. After 2 days, it was replaced with a medium containing insulin, and it was replaced with a new medium again per 3 days to induce differentiation.

(4) Oil Red O Staining

The differentiation-completed 3T3-L1 cells were fixed with 4% formaldehyde for 1 hour, and then fats were stained using a 0.5% Oil red O staining solution to confirm the degree of differentiation. For quantitative analysis of adipose differentiation, the dye stained in the cells was dissolved with 100% isopropyl alcohol and the absorbance was measured at 500 nm.

(5) Western Blotting

In order to investigate the effect on expression of the adipocytes differentiation-related proteins by the sea cucumber extract, expression of the related proteins was confirmed through western blot. The differentiation-completed 3T3-L1 cells were washed with PBS, and then lysis was conducted with RIPA buffer, and then the proteins were separated by molecular weight through SDS-PAGE. After that, it was transferred to a PVDF membrane and it was blocked using 5% skim milk for 30 minutes. Then, it was reacted with a primary antibody, and then a secondary antibody was attached to confirm protein expression using an ECL solution.

(6) Statistics

Statistical analysis of the result was conducted using GraphPad Prism 7 software (San Diego, CA, USA), and the significant difference between each group was analyzed through One-way ANOVA. (p<0.05)

2. Experimental Result

(1) Process of Preparing Extracts and Fractions

Ethanol extracts were obtained by extracting intestinal organs except for an ovary and an ovary of a sea cucumber as follows, respectively. After 30% EtOH, 50% EtOH or 80% EtOH was added to the dried sea cucumber ovary 20 times, they were stirred for 24 hours, and then filtered and concentrated in the same manner.

Fractions of the sea cucumber ovary extract were obtained by the following method.

After suspending the concentrated sea cucumber ovary extract by adding distilled water, the same amount of n-hexane was added and shaken and then stood. After completing layer separation, the n-hexane layer was separated and concentrated. Water saturated butanol was added to the remaining suspension and shaken, and then stood sufficiently, and after the layer separation was completed, the water saturated butanol layer was separated and concentrated.

(2) Oil Red O Staining Result

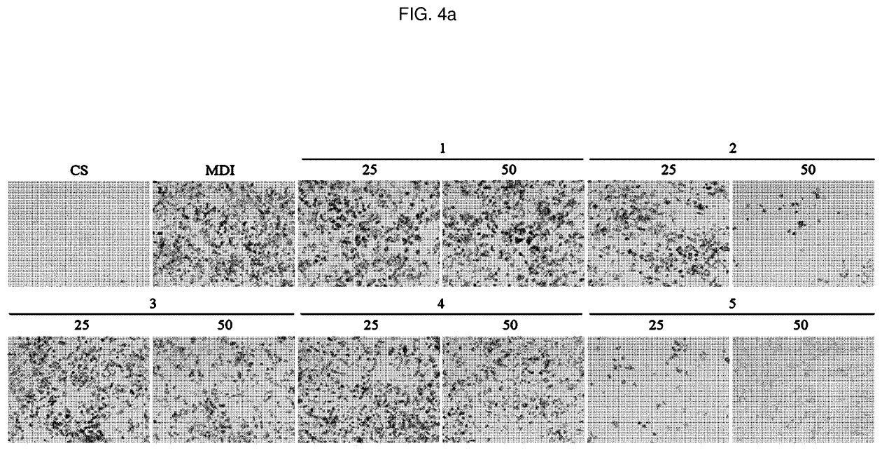

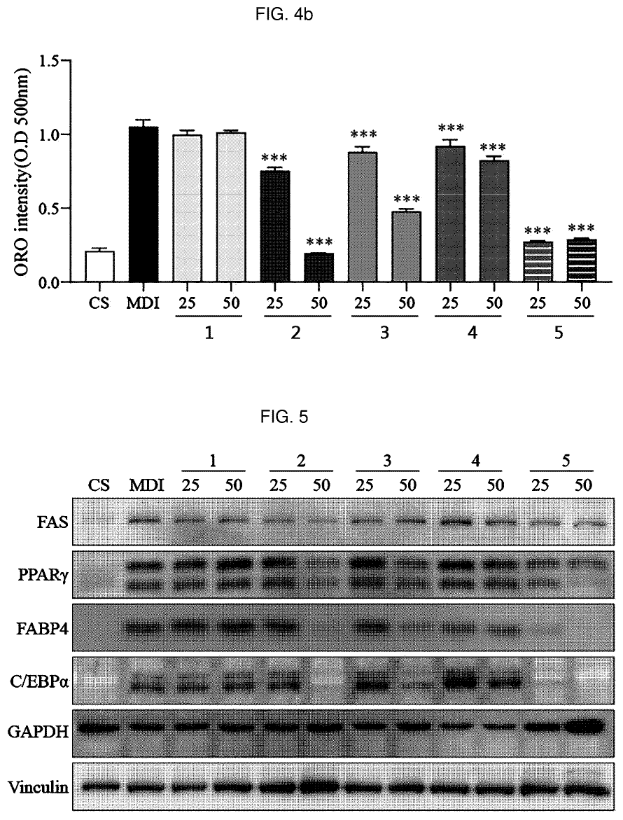

In order to investigate the effect on adipose differentiation of the 3T3-L1 cells of the sea cucumber extract, each sea cucumber extract was treated with a differentiation medium at a concentration of 25 and 50 μg/ml (Sample No. 6, 10, 25 μg/ml) for 48 hours to proceed an experiment. As a result of analyzing the degree of fat accumulation through Oil red O staining, all the samples except for Sample No. 1 showed a significant effect of inhibiting fat accumulation. (FIG. 4)

(3) Analysis of Adipocyte Differentiation, Lipids in Blood, and Inflammation-Related Proteins

Various proteins are involved in the process of adipocyte differentiation, and when these proteins are inhibited, adipose differentiation and accumulation are inhibited. As a result of confirming the effect of the sea cucumber extract on expression of major related proteins, PPARγ, FAS, aP2, and C/EBPα, similarly to the previous experiment, it could be confirmed that expression of adipose differentiation-related proteins was reduced in five samples except for No. 1 sample in two independent experiments, and accordingly, taking these results together, it can be seen that the sea cucumber extract inhibits expression of proteins related to adipose differentiation and this inhibitory effect inhibits adipose differentiation and accumulation.

Through the above experiment, as a result of experimenting an effect of inhibiting adipocytes differentiation and fat accumulation of the sea cucumber extract extracted under each condition, a concentration-dependent effect of inhibiting fat accumulation was confirmed at a concentration of 25 and 50 μg/ml. It seems that this effect inhibits adipocyte differentiation and expression of fat accumulation-related proteins such as PPARγ, FAS, aP2, and C/EBPα and regulates a process of differentiation into adipocytes, and it seems that the sea cucumber extract has a significant effect of inhibiting adipocyte differentiation. In case of PPARγ, it is a factor having a correlation with inflammatory disease, and in case of FAS, it is an enzyme involved in fatty acid biosynthesis, and it has been known as an enzyme which regulates a concentration of triglyceride in blood and SREBP-1 (transcription factor regulating synthesis of cholesterol and fatty acids in fats and liver tissue). Therefore, through inhibition of expression of these proteins, an effect of improving dyslipidemia and inflammation can be obtained.

### III. Experimental Example 3—Effect of Compound Separated from Sea Cucumber Ovary Extract

1. Separation, Structure Identification of 5 Kinds of Novel Saponins and 3 Kinds of Conventionally Reported Saponin Compounds

Compounds corresponding to Chemical formulas S1, S2, S3, S4, S5, S6, S8 and S9 were separated from butanol fractions of the sea cucumber ovary extract by the following method. In order to isolate active components from the extract, separation and purification were performed according to activity guided fractionation. In order to separate active components from water saturated butanol fractions of the sea cucumber ovary, 8 kinds of saponin components were separated/purified by conducting various kinds of column chromatography.

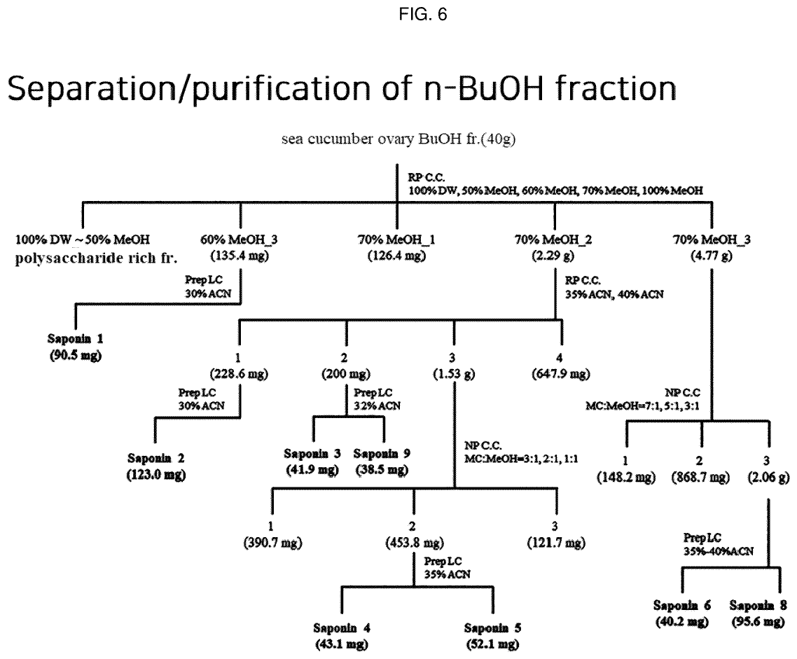

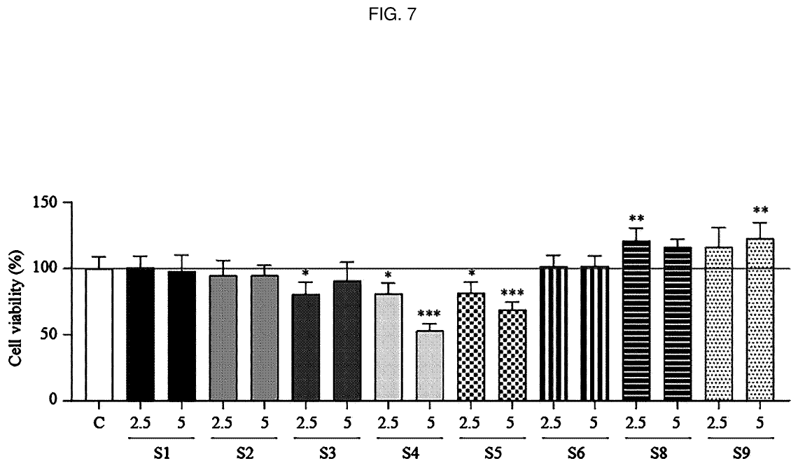

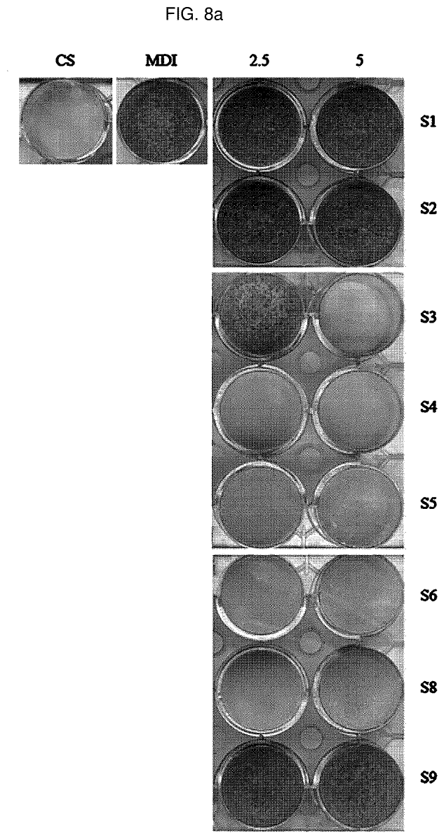

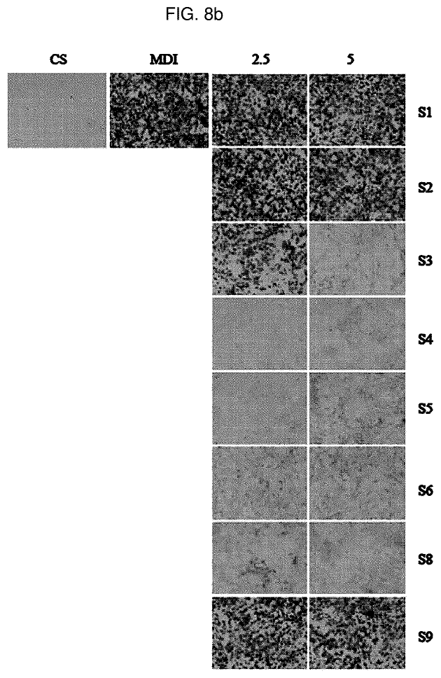

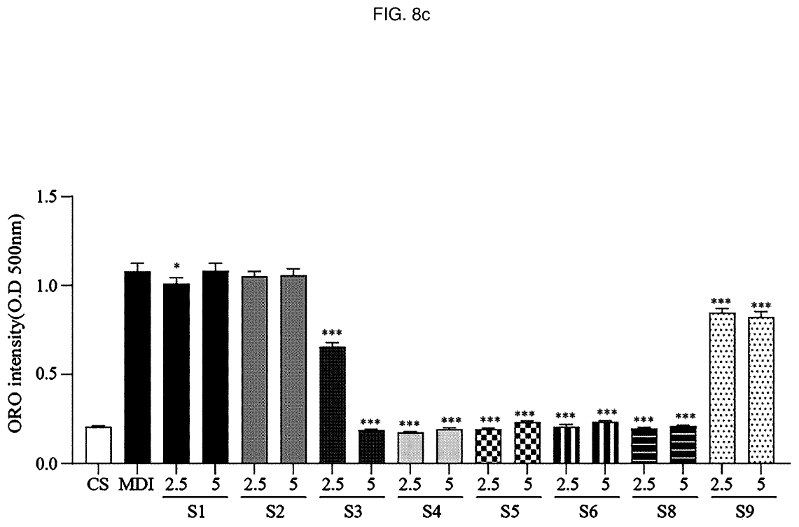

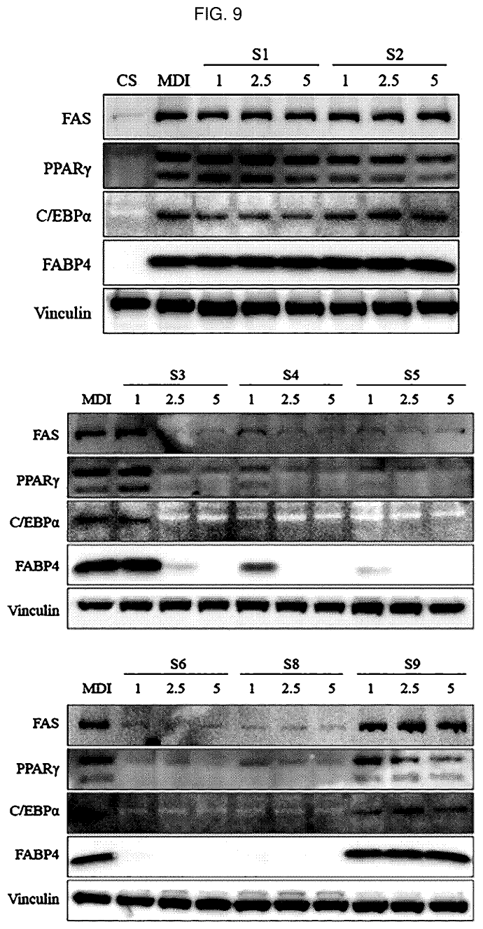

The separation/purification method was shown in FIG. 6.

The specific separation/purification method is as follows.
1) The sea cucumber ovary butanol fractions obtained from the above were divided into small fractions through reversed-phase column chromatography.
2) polysaccharides were removed by erupting with 50% methanol in 100% distilled water, and after that, 60% methanol and 70% methanol fractions 1 and 2 were obtained, respectively.
3) The 60% methanol fraction was erupted with 30% ACN through preparative LC to obtain Saponin 1.
4) The 60% methanol fraction was erupted with 30% ACN through preparative LC to obtain Saponin 1.
5) The 70% methanol fraction 1 was divided into 4 small fractions through reversed-phase column chromatography again, and among them, No. 1 small fraction was erupted with 30% ACN through preparative LC to obtain Saponin 2.
6) The above No. 2 small fraction was erupted with 32% ACN through preparative LC to obtain Saponins 3 and 9, respectively.
7) The above No. 3 small fraction was divided into MC:MeOH=3:1, 2:1, 1:1 fractions through normal phase column chromatography again, and among then, the MC:MeOH=2:1 fraction was erupted with 35% ACN through preparative LC to obtain Saponins 4 and 5.
8) The 70% methanol fraction 2 was divided into MC:MeOH=7:1, 5:1. 3:1 fractions through normal phase column chromatography again, and among them, the MC:MeOH=3:1 fraction was erupted with 35%-40% ACN through preparative LC to obtain Saponins 6 and 8, respectively.

Among them, the compounds corresponding to Chemical formulas S1, S2, S3, S4 and S9 corresponding to saponin-1, 2, 3, 4, and 9, respectively, were confirmed as novel compounds, and Saponin-5, 6, and 8 were confirmed as a chemical structure of holotoxin D1, holotoxin B, and holotoxin A, respectively.

2. Experimental Result

(1) Cell Viability

In order to investigate the effect on the 3T3-L1 cell viability of a total 8 kinds of the single compounds, as a result of confirming the cell viability by treating each of the single compounds at a concentration of 2.5-5 μg/ml, it could be confirmed that a significant change was not shown in a majority of samples. In the samples of S3, S4, and S5, a significant decrease was shown at a concentration of 2.5 μg/ml, and the cell viability of about 80% or more was shown.

(2) Oil Red O Staining Result

In order to investigate the effect on adipose differentiation of the 3T3-L1 cells of the single compound in the sea cucumber ovary extract, a total 8 of compounds were treated with a differentiation medium at a concentration of 2.5 and 5 μg/ml for 48 hours. After completing differentiation, it could be confirmed that a significant difference was not shown at all the concentrations, but when S3-S9 were treated, fat accumulation was significantly reduced at a concentration of 2.5, 5 μg/ml. It could be confirmed that the sample of S3 inhibited fat accumulation of about 16, 40, 80% from 1 μg/ml to 5 μg/ml and showed a tendency to have adipose differentiation inhibitory efficacy in a concentration dependent manner, and samples of S4-S8 inhibited fat accumulation at a level of non-differentiated cells at 2.5 μg/ml.

(3) Analysis of Adipocyte Differentiation-Related Proteins

In order to figure out the mechanism of the fat accumulation inhibitory efficacy of the single compound examined above, the degree of expression of proteins of FAS, PPARγ, C/EBPα, and FABP4, which were adipogenesis-related proteins was confirmed. As a result, it could be confirmed that in the 3T3-L1 cells treated with S2, S2, compared to the control in which anything was not treated, a significant difference in protein expression was not shown, but in the cells treated with S3, S4, S5, from the cell treated with 2.5 μg/ml, expression of the related proteins was definitely reduced, and it could be confirmed that it was definitely reduced in the cells treated with 1 μg/ml compared to the control group. Next, as a result of confirming protein expression in the cells treated with S6, S8, S9, compared to the control group, a definite decrease in protein expression could be confirmed in S6 and S8. Therefore, taking these results together, it seems that the single compounds, S3-6, S8 inhibits adipocyte differentiation and fat accumulation through inhibition of expression of adipogenesis-related proteins.

Through the experiment at this time, an effect of inhibiting adipocyte differentiation and fat accumulation of the single compound in the sea cucumber ovary extract was confirmed. As the experimental result of the previous extract, it seemed that expression of FAS, PPARγ, C/EBPα, and FABP4, which are adipogenesis-related proteins, was significantly reduced, and it inhibited adipocyte differentiation through such protein inhibition.

<Nmr Data>

Tables 4 to 9 below show the result of ¹³C and ¹H NMR chemical shifts of S1-S4 and S9 among the compounds separated from the sea cucumber ovary extract. Through this, it was confirmed that S1-S4 and S9 were newly separated saponin compounds.

TABLE 4
¹³C NMR (200 MHz) data for aglycones of compounds 1-3, 9 in
C₅D₅N/D₂O(δ in ppm)

| Position | 1 | 2 | 3 | 9 |
|---|---|---|---|---|
| 1 | 36.0 | 36.0 | 36.0 | 36.0 |
| 2 | 27.1 | 27.2 | 27.2 | 27.2 |
| 3 | 89.0 | 89.1 | 89.1 | 89.1 |
| 4 | 39.5 | 39.6 | 39.6 | 39.6 |
| 5 | 47.7 | 47.8 | 47.8 | 47.8 |
| 6 | 23.4 | 23.5 | 23.5 | 23.4 |
| 7 | 122.6 | 122.6 | 122.6 | 122.6 |
| 8 | 147.9 | 148.0 | 148.0 | 148.0 |
| 9 | 46.2 | 46.2 | 46.2 | 46.2 |
| 10 | 35.7 | 35.7 | 35.7 | 35.7 |
| 11 | 22.1 | 22.1 | 22.1 | 22.0 |
| 12 | 20.6 | 20.6 | 20.6 | 20.6 |
| 13 | 54.8 | 54.9 | 54.9 | 54.9 |
| 14 | 46.0 | 46.0 | 46.1 | 46.1 |
| 15 | 44.7 | 44.7 | 44.7 | 44.7 |
| 16 | 79.8 | 79.9 | 79.9 | 79.9 |
| 17 | 62.5 | 62.5 | 62.5 | 62.5 |
| 18 | 182.1 | 182.2 | 182.2 | 182.3 |
| 19 | 24.1 | 24.1 | 24.2 | 24.1 |
| 20 | 71.3 | 71.3 | 71.3 | 71.3 |
| 21 | 26.7 | 26.7 | 26.7 | 26.6 |
| 22 | 42.6 | 42.5 | 42.5 | 42.5 |
| 23 | 22.1 | 22.1 | 22.1 | 22.1 |
| 24 | 38.5 | 38.5 | 38.5 | 38.5 |
| 25 | 146.0 | 146.0 | 146.1 | 146.0 |
| 26 | 110.5 | 110.5 | 110.5 | 110.6 |
| 27 | 22.4 | 22.4 | 22.4 | 22.5 |
| 30 | 17.4 | 17.3 | 17.3 | 17.3 |
| 31 | 28.8. | 28.7 | 28.7 | 28.7 |
| 32 | 34.6 | 34.6 | 34.6 | 34.6 |

TABLE 5
¹H NMR (800 MHz) data for aglycones of compounds 1-3, 9 in
C₅D₅N/D₂O(δ in ppm)

| Position | 1 | 2 | 3 | 9 |
|---|---|---|---|---|
| 1 | 1.47 (2H) | 1.47 (2H) | 1.47 (2H) | 1.47 (2H) |
| 2 | 2.09, 1.88 | 2.09, 1.88 | 2.10, 1.87 | 2.10, 1.87 |
| 3 | 3.25 | 3.26 | 3.25 | 3.25 |
| 4 | — | — | — | — |
| 5 | 0.98 | 0.97 | 0.99 | 0.98 |
| 6 | 2.05, 1.97 | 2.04, 1.96 | 2.05, 1.97 | 2.04, 1.96 |
| 7 | 5.67 | 5.66 | 5.67 | 5.67 |
| 8 | — | — | — | — |
| 9 | 3.25 | 3.25 | 3.23 | 3.24 |
| 10 | — | — | — | — |
| 11 | 2.05, 1.84 | 2.05, 1.83 | 2.05, 1.84 | 2.05, 1.87 |
| 12 | 2.67, 2.18 | 2.66, 2.17 | 2.66, 2.18 | 2.66, 2.19 |
| 13 | — | — | — | — |
| 14 | — | — | — | — |
| 15 | 2.17, 2.03 | 2.18, 2.01 | 2.19, 2.03 | 2.19, 2.02 |
| 16 | 5.11 | 5.10 | 5.11 | 5.12 |
| 17 | 2.69 | 2.67 | 2.69 | 2.70 |
| 18 | — | — | — | — |
| 19 | 1.06 (3H) | 1.05 (3H) | 1.06 (3H) | 1.05 (3H) |
| 20 | — | — | — | — |
| 21 | 1.52 (3H) | 1.51 (3H) | 1.52 (3H) | 1.52 (3H) |
| 22 | 1.82, 1.78 | 1.82, 1.77 | 1.82, 1.78 | 1.82, 1.78 |
| 23 | 1.72, 1.53 | 1.72, 1.53 | 1.71, 1.53 | 1.72, 1.53 |
| 24 | 2.08 (2H) | 2.07 (2H) | 2.08 (2H) | 2.08 (2H) |
| 25 | — | — | — | — |
| 26 | 4.83 (2H) | 4.83 (2H) | 4.83 (2H) | 4.83 (2H) |
| 27 | 1.73 (3H) | 1.73 (3H) | 1.73 (3H) | 1.73 (3H) |
| 30 | 1.11 (3H) | 1.11 (3H) | 1.11 (3H) | 1.10 (3H) |
| 31 | 1.28 (3H) | 1.28 (3H) | 1.28 (3H) | 1.27 (3H) |
| 32 | 1.44 (3H) | 1.43 (3H) | 1.45 (3H) | 1.45 (3H) |

TABLE 6
¹³C NMR (200 MHz) data for glycoside moiety of compounds 1-3, 9 in C₅D₅N/D₂O(δ in ppm)

| Position | 1 | 2 | 3 | 9 |
|---|---|---|---|---|
| | Xy1¹(1→C3) | Xy1¹(1→C3) | Xy1¹(1→C3) | Xy1¹(1→C3) |
| 1 | 105.3 | 105.2 | 105.2 | 105.2 |
| 2 | 82.6 | 83.4 | 83.3 | 83.3 |
| 3 | 75.7 | 75.8 | 75.7 | 75.8 |
| 4 | 77.6 | 77.4 | 77.4 | 77.4 |
| 5 | 64.1 | 64.1 | 64.1 | 64.1 |
| | Glc¹(1→4Xyl¹) | Glc¹(1→4Xyl¹) | Glc¹(1→4Xyl¹) | Glc¹(1→4Xyl¹) |
| 1 | 103.4 | 103.3 | 103.3 | 103.3 |
| 2 | 74.4 | 74.3 | 74.4 | 74.4 |
| 3 | 78.0 | 78.0 | 78.0 | 77.9 |
| 4 | 71.5 | 71.5 | 71.5 | 71.4 |
| 5 | 78.7 | 78.6 | 78.3 | 78.6 |
| 6 | 62.4 | 62.4 | 62.4 | 62.3 |
| | Glc²(1→2Xyl¹) | Quin¹(1→2Xyl¹) | Quin¹(1→2Xyl¹) | Quin¹(1→2Xyl¹) |
| 1 | 105.4 | 105.4 | 105.4 | 105.4 |
| 2 | 76.2 | 76.2 | 76.2 | 76.2 |
| 3 | 75.9 | 75.7 | 75.8 | 75.8 |
| 4 | 81.5 | 87.3 | 87.2 | 87.1 |
| 5 | 76.6 | 71.7 | 71.7 | 71.7 |
| 6(or Me) | 61.5 | 18.2 | 18.2 | 18.3 |
| | Glc³(1→4Glc²) | Glc²(1→4Quin¹) | Glc²(1→4Quin¹) | Glc²(1→4Quin¹) |
| 1 | 104.4 | 104.8 | 104.8 | 105.3 |
| 2 | 73.7 | 73.8 | 73.8 | 74.8 |
| 3 | 88,0 | 88.0 | 87.7 | 78.0 |
| 4 | 69.7 | 69.8 | 69.8 | 71.5 |
| 5 | 77.9 | 77.9 | 77.9 | 78.4 |
| 6 | 62.1 | 62.2 | 62.1 | 62.4 |
| | Glc⁴(1→3Glc²) | Glc³(1→3Glc²) | MeGlc¹(1→3Glc²) | — |
| 1 | 105.6 | 105.6 | 105.3 | — |
| 2 | 75.4 | 75.4 | 75.0 | — |
| 3 | 78.0 | 78.0 | 87.8 | — |
| 4 | 71.5 | 71.5 | 70.6 | — |
| 5 | 78.6 | 78.5 | 78.6 | — |
| 6 | 62.4 | 62.4 | 62.1 | — |
| Me | — | — | 60.8 | — |

TABLE 7
¹H NMR (800 MHz) data for glycoside moiety of compounds 1-3, 9 in C₅D₅N/D₂O(δ in ppm)

| Position | 1 | 2 | 3 | 9 |
|---|---|---|---|---|
| | Xy1¹(1→C3) | Xy1¹(1→C3) | Xy1¹(1→C3) | Xy1¹(1→C3) |
| 1 | 4.73 | 4.72 | 4.73 | 4.73 |
| 2 | 4.14 | 4.04 | 4.04 | 4.04 |
| 3 | 4.26 | 4.24 | 4.25 | 4.25 |
| 4 | 4.29 | 4.33 | 4.33 | 4.33 |
| 5 | 4.43, 3.66 | 4.43, 3.66 | 4.44, 3.67 | 4.44, 3.67 |
| | Glc¹(1→4Xyl¹) | Glc¹(1→4Xyl¹) | Glc¹(1→4Xyl¹) | Glc¹(1→4Xyl¹) |
| 1 | 5.02 | 5.02 | 5.02 | 5.02 |
| 2 | 4.03 | 4.03 | 4.02 | 4.03 |
| 3 | 4.25 | 4.25 | 4.24 | 4.26 |
| 4 | 4.18 | 4.16 | 4.15 | 4.15 |
| 5 | 4.01 | 4.00 | 4.00 | 4.01 |
| 6 | 4.55, 4.31 | 4.55, 4.29 | 4.54, 4.27 | 4.55, 4.28 |
| | Glc²(1→2Xyl¹) | Quin¹(1→2Xyl¹) | Quin¹(1→2Xyl¹) | Quin¹(1→2Xyl¹) |
| 1 | 5.26 | 5.13 | 5.13 | 5.13 |
| 2 | 4.05 | 4.03 | 4.03 | 4.04 |
| 3 | 4.22 | 4.10 | 4.10 | 4.12 |
| 4 | 4.33 | 3.66 | 3.66 | 3.67 |
| 5 | 3.86 | 3.77 | 3.76 | 3.77 |
| 6(or Me) | 4.55, 4,43 | 1.74 | 1.74 | 1.74 |
| | Glc³(1→4Glc²) | Glc²(1→4Quin¹) | Glc²(1→4Quin¹) | Glc²(1→4Quin¹) |
| 1 | 5.16 | 4.97 | 4.97 | 4.98 |
| 2 | 4.09 | 4.07 | 4.05 | 4.04 |
| 3 | 4.21 | 4.27 | 4.29 | 4.27 |
| 4 | 4.05 | 4.02 | 4.03 | 4.13 |
| 5 | 3.96 | 4.02 | 4.03 | 4.09 |
| 6 | 4.45, 4.17 | 4.50, 4.17 | 4.50, 4.17 | 4.61, 4.25 |
| | Glc⁴(1→3Glc²) | Glc³(1→3Glc²) | MeGlc¹(1→3Glc²) | — |
| 1 | 5.28 | 5.33 | 5.33 | — |
| 2 | 4.07 | 4.08 | 4.00 | — |
| 3 | 4.25 | 4.25 | 3.75 | — |
| 4 | 4.15 | 4.14 | 4.08 | — |
| 5 | 4.03 | 4.04 | 4.01 | — |
| 6 | 4.55, 4.27 | 4.55, 4.26 | 4.51, 4.23 | — |
| Me | — | — | 3.89 | — |

TABLE 8
¹³C and ¹H NMR data for aglycone moiety of compound 4 in
C₅D₅N/D₂O(δ in ppm)

| | δ C^{a)} | δ H^{b)} |
|---|---|---|
| 1 | 36.3 | 1.84, 1.42 |
| 2 | 27.0 | 2.20, 2.01 |
| 3 | 88.9 | 3.24 |
| 4 | 39.9 | — |
| 5 | 52.8 | 0.87 |
| 6 | 21.1 | 1.62, 1.46 |
| 7 | 28.5 | 1,62, 1.21 |
| 8 | 38.8 | 3.24 |
| 9 | 151.2 | — |
| 10 | 39.7 | — |
| 11 | 111.2 | — |
| 12 | 32.1 | 2.65, 2.55 |
| 13 | 55.9 | — |
| 14 | 42.1 | — |
| 15 | 52.1 | 2.45, 2.22 |
| 16 | 214.0 | — |
| 17 | 61.3 | 2.94 |
| 18 | 176.5 | — |
| 19 | 22.1 | 1.37 (3H) |
| 20 | 83.5 | — |
| 21 | 26.9 | 1.49 (3H) |
| 22 | 38.4 | 1.82, 1.66 |
| 23 | 22.3 | 1.81, 1.54 |
| 24 | 38.0 | 2.00, 1.97 |
| 25 | 145.6 | — |
| 26 | 110.6 | 4.79 (2H) |
| 27 | 22.3 | 1.71 (3H) |
| 30 | 16.7 | 1.11 (3H) |
| 31 | 28.1 | 1.23 (3H) |
| 32 | 20.7 | 0.95 (3H) |

TABLE 9
¹³C and ¹H NMR data for glycoside moiety of compound 4 in
C₅D₅N/D₂O(δ in ppm)

| | 8 C^{a)} | 8 H^{b)} |
|---|---|---|
| | Xy1¹(1→C3) | Xy1¹(1→C3) |
| 1 | 105.2 | 4.78 |
| 2 | 81.9 | 4.20 |
| 3 | 75.5 | 4.29 |
| 4 | 77.7 | 4.29 |
| 5 | 63.9 | 63.9 |
| | Glc¹(1→2Xyl¹) | Glc¹(1→2Xyl¹) |
| 1 | 104.9 | 5.30 |
| 2 | 75.9 | 4.05 |
| 3 | 75.8 | 4.20 |
| 4 | 81.4 | 81.4 |
| 5 | 76.5 | 76.5 |
| 6 (or Me) | 61.5 | 61.5 |
| | Glc²(1→4Glc¹) | Glc²(1→4Glc¹) |
| 1 | 104.1 | 5.14 |
| 2 | 73.8 | 4.08 |
| 3 | 87.6 | 4.25 |
| 4 | 69.5 | 3.99 |
| 5 | 77.6 | 3.98 |
| 6 | 61.9 | 4.44, 4.15 |
| | Glc³(1→3Glc²) | Glc³(1→3Glc²) |
| 1 | 105.2 | 5.28 |
| 2 | 75.3 | 4.07 |
| 3 | 77.7 | 4.25 |
| 4 | 71.4 | 4.09 |
| 5 | 78.3 | 4.04 |
| 6 | 62.3 | 4.55, 4.22 |
| Me | | |
| | Glc⁴(1→4Xyl¹) | Glc⁴(1→4Xyl¹) |
| 1 | 102.6 | 5.01 |
| 2 | 73.5 | 4.03 |
| 3 | 87.4 | 4.31 |
| 4 | 69.6 | 69.6 |
| 5 | 77.8 | 77.8 |
| 6 | 61.9 | 61.9 |
| | MeGlc¹(1→3Glc⁴) | MeGlc¹(1→3Glc⁴) |
| 1 | 105.1 | 5.33 |
| 2 | 74.9 | 3.99 |
| 3 | 87.5 | 3.77 |
| 4 | 70.6 | 1.04 |
| 5 | 78.1 | 4.04 |
| 6 | 62.1 | 4.52. 4.20 |
| Me | 60.9 | 3.90 |

a) Measured at 200 MHz in C₅D₅N/D₂O, b) Measured at 800 MHz in C₅D₅N/D₂O.

Through the NMR data, it was confirmed that S1-S4 and S9 were novel saponin compounds.

### IV. Experimental Example 4—Confirmation of Anti-Diabetic Effect of Sea Cucumber Ovary Extract

1. Experimental Materials and Methods

(1) Reagents and Materials

3T3-L1 cells (ATCC, CL-173), DMEM high glucose (Welgene), Fetal bovine serum (MPBio), penicillin-streptomycin (Welgene), Dulbecco's phosphate-buffered saline (Welgene), Trypsin-EDTA (Welgene), Bovine calf serum (Welgene), Glucose Uptake-Glo Assay Kit (Promega)

(2) Culture of 3T3-L1 Preadipocytes and Differentiation of Adipocytes

The 3T3-L1 cells were cultured in a high glucose DMEM medium in which 10% Calf serum and penicillin-streptomycin were added, and subcultured per 2-3 days. The 3T3-L1 cells were aliquoted in a 96 well plate at a concentration of 2×10⁴ cells/well and then cultured for 3 days, and then it was replaced with an MDI medium that dexamethasone, insulin and 3-isobuty-1-methylxanthine (IBMX) were added to a medium containing 10% PBS instead of calf serum. After 2 days, it was replaced with a medium containing insulin, and the medium was replaced again per 2 days and differentiation was induced.

(3) Glucose Uptake-Glo Assay

The differentiation-completed 3T3-L1 cells were under starvation for 16 hours, and then the sea cucumber extract was treated in a serum-free medium at a concentration of 25, 50 ug/mL for 6 hours. The cultured supernatant was recovered and in the 3T3-L1 cells, using Glucose Uptake-Glo Assay Kit, a glucose transport activity degree was measured using a luminometer.

2. Experimental Result

(1) Result of Confirming Degree of Glucose Uptake into Cells (Muscle Glucose Uptake) of Sea Cucumber Ovary Extract

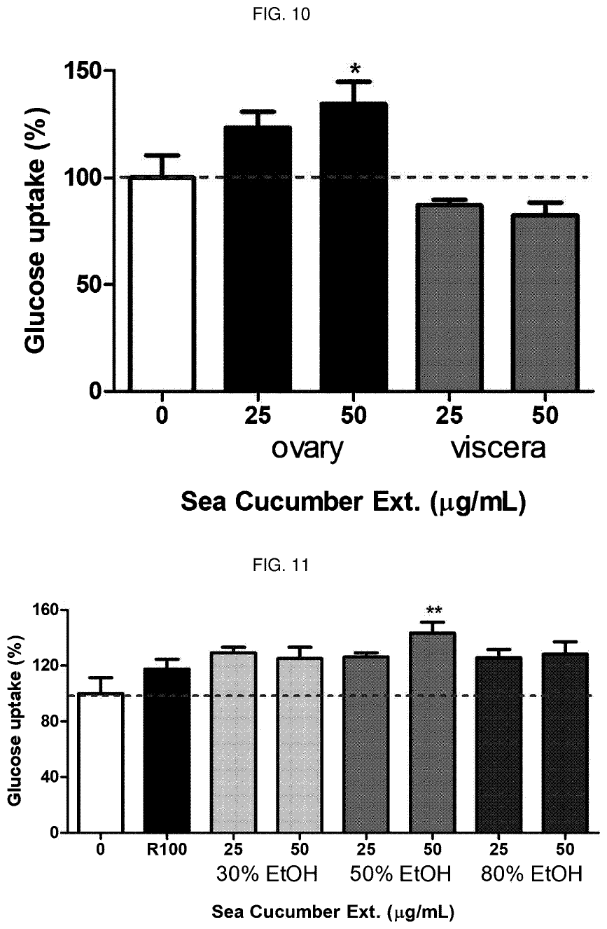

As can be confirmed in FIG. 10, the intracellular glucose uptake of the sea cucumber ovary extract is significantly excellent. FIG. 10 is the result of confirming the glucose uptake of the sea cucumber ovary extract and extract of intestinal organs except for an ovary of a sea cucumber. FIG. 11 is the result of confirming the glucose uptake by extraction condition of the sea cucumber ovary extract. At all the concentrations, the glucose uptake was excellent compared to the control group. In particular, it could be seen that the sea cucumber ovary extract had an excellent anti-diabetic effect, as it had glucose uptake efficiency increased more than Rosiglitazone conventionally used as a therapeutic agent for diabetes.

As can be confirmed in Table 10 below, the sea cucumber ovary extract had the glucose uptake increased compared to the untreated group, and in particular, the sea cucumber ovary extract showed the uptake at a much higher level (1.29-fold, 1.43-fold, 1.35-fold) at the entire concentrations compared to the 1.24-fold increase of Rosiglitazone.

TABLE 10

| Sample | Fold change |
|---|---|
| Rosiglitazone (Ref.) | 1.24 |
| 30% EtOH | ~1.29 |
| 50% EtOH | ~1.43 |
| 80% EtOH | ~1.35 |

Through the result, it was confirmed that it had increased glucose uptake by treatment of the sea cucumber ovary extract.

### V. Experimental Example 5—Confirmation of Anti-Diabetic Effect of Saponin Compounds Separated from Sea Cucumber Ovary Extract

1. Experimental Method

(1) Reagents and Materials

They were prepared as the items of IV-1-(1).

(2) Culture of 3T3-L1 Preadipocytes and Differentiation of Adipocytes

The 3T3-L1 cells were cultured in a high glucose DMEM medium in which 10% Calf serum and penicillin-streptomycin were added, and subcultured per 2-3 days. The 3T3-L1 cells were aliquoted in a 96 well plate at a concentration of 2×10⁴ cells/well and cultured for 3 days, and then it was replaced with an MDI medium that dexamethasone, insulin and 3-isobuty-1-methylxanthine (IBMX) were added to a medium containing 10% FBS instead of calf serum. After 2 days, it was replaced with a medium containing insulin, and the medium was replaced again per 2 days to induce differentiation.

(3) Glucose Uptake-Glo Assay

For the differentiation-completed 3T3-L1 cells, starvation was conducted for 16 hours, and then a sea cucumber-derived single compound was treated in a serum-free medium at a concentration of 2.5 ug/mL for 6 hours. The cultured supernatant was recovered and for the 3T3-L1 cells, using Glucose Uptake-Glo Assay Kit, the glucose transport activity degree was measured using a luminometer.

For the saponin compounds obtained previously, the anti-diabetic effect was confirmed.

2. Experimental Result

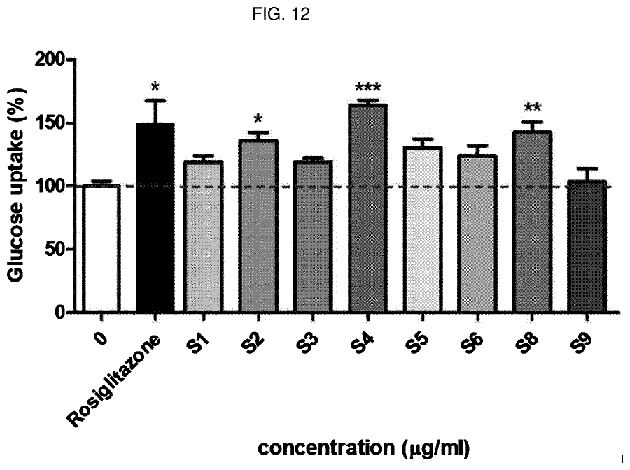

FIG. 12 is the result of confirming the glucose uptake of the saponin compounds separated from the sea cucumber ovary extract. As could be confirmed in FIG. 12, when the glucose uptake of the untreated group was considered as 100%, all the compounds showed the uptake of 100% or more. Through this result, it could be seen that the separated compounds increased the glucose uptake compared to the untreated group, and it could be seen that each had an anti-diabetic effect.

Table 11 below is fold change values, showing the degree of the glucose uptake compared to the control group. A glucose uptake at a similar level to Rosiglitazone was also shown.

TABLE 11

| Sample | Fold Relative to Control |
|---|---|
| Rosiglitazone (Ref.) | 1.49 |
| S1 | 1.25 |
| S2 | 1.43 |
| S3 | 1.1 |
| S4 | 1.6 |
| S5 | 1.38 |
| S6 | 1.24 |
| S8 | 1.43 |
| S9 | 1.03 |

### VI. Experimental Example 6—In Vivo Experiment of Sea Cucumber Ovary Extract

[Effect of Reducing Body Weight (Anti-Obesity Use)]

1. Experimental Method

(1) Experimental Animal Group Information

An experiment was performed for C57BL/6 male mice (5-week-old), for a total of 50 animals, 10 in each group. In all other groups except for the normal diet group, a high-fat diet was fed.
1) Normal (Normal diet group)
2) HFD (High fat diet group)
3) HFD+0.05% sea cucumber ovary extract (SCOE)
4) HFD+0.1% sea cucumber ovary extract (SCOE)
5) HFD+0.2% sea cucumber ovary extract (SCOE)

(2) Breeding Condition

The extract was fed through a diet by preparing a feed, and an experiment was carried out for 8 weeks. The body weight was measured once a week, and the diet intake was measured 3 times a week.

2. Experimental Result

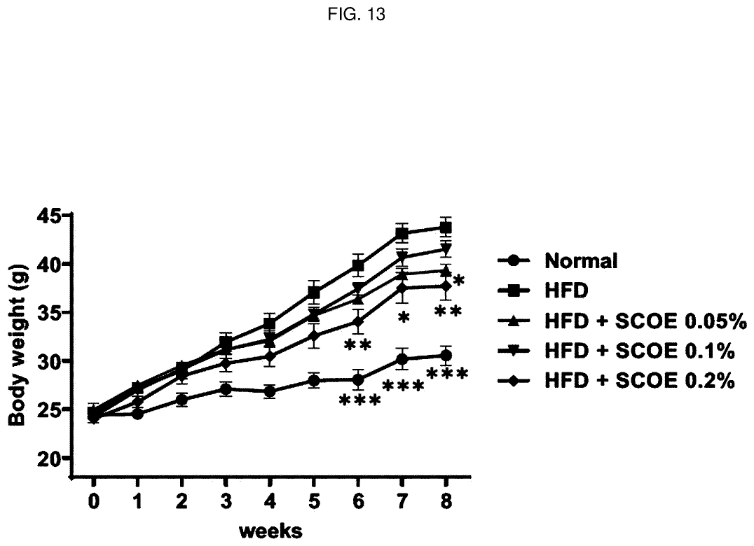

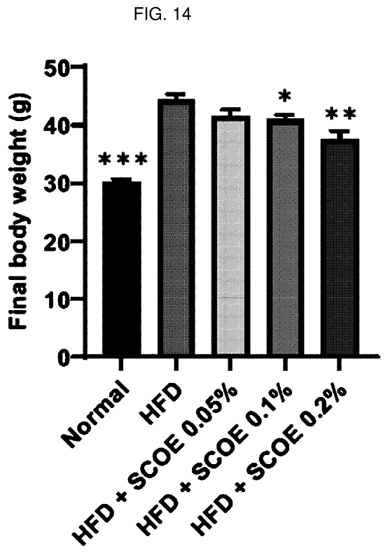

FIGS. 13 and 14 are results which observe the change in body weight and the finally confirmed body weight of the high calorie diet intake mice. Through FIG. 13, it could be seen that in the group treated with the sea cucumber ovary extract (50% ethanol), the body weight increase rate was significantly reduced as a result of confirming up to 8 weeks. As confirmed through FIG. 14, it could be seen that the final body weight was reduced, when compared to the untreated group with the sea cucumber ovary extract indicated by HFD.

[White Adipose Tissue Reducing Effect (Anti-Obesity Use)]

1. Experimental Method

(1) H&E Stain of Adipose Tissue

Hematoxilin is a basic/cationic substance, which stains the nucleus (anion) in dark blue or purple, and eosin is an acidic/anionic substance, which stains the cytoplasm (cation) in red or pink.

The process of H&E staining is as follows.

Dewaxing→dehydration→hematoxylin→differentiation→blueing→eosin→dehydration→clearing→cover-slipping

(2) White fats among adipose tissue are related to obesity and overweight, and in the present experiment, lipid accumulation of epididymis white fats was histologically analyzed by H&E staining to confirm the tissue size of the white adipose tissue.

2. Result

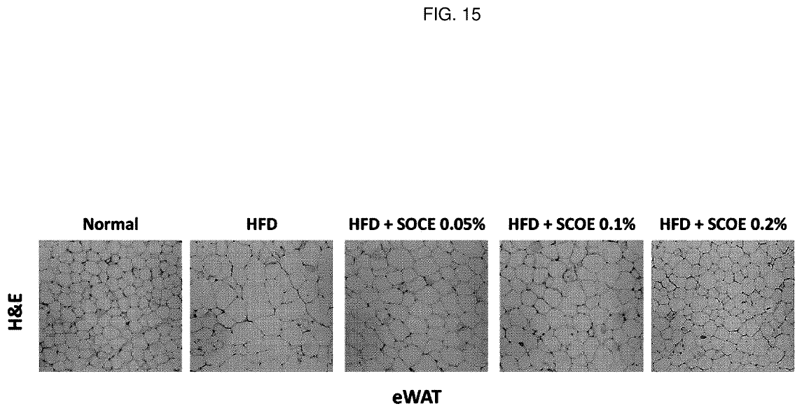

FIG. 15 is the result of the change in the white adipocyte confirmed through H&E stain.

It was confirmed that when a high calorie diet was administered (HFD), the size of the white adipocyte related to obesity became significantly larger, and when the sea cucumber ovary extract was administered herein (HFD+SOCE 0.05%, HFD+SOCE 0.1%, HFD+SOCE 0.2%), the adipocyte size was significantly reduced. Through this, it was confirmed that an adipocyte size decrease, an anti-obesity effect, and the like could be obtained by treatment of the sea cucumber ovary extract.

[Confirmation of Decrease in Total Cholesterol and Triglyceride in Blood (Improvement of Hyperlipidemia, Metabolic Syndrome)]

1. Experimental Method

(1) High-Density Lipoprotein Content Compared to Total Cholesterol in Blood (HDL/TC)

A sinking reagent of 200 μl was added to a blood sample of 200 μl, and then they were mixed and left at a room temperature for 5 minutes. After 5 minutes, centrifugation was performed at 3000 rpm for 10 minutes, and an enzyme solution of 3 ml was added to the centrifuged supernatant of 100 μl each, and mixed. They were reacted in a 37° C. constant-temperature water bath for 5 minutes. Using a blind test as a control, the absorbance was measured at a wavelength of 500 nm.

HDL-C(mg/dl)=(absorbance of specimen/absorbance of standard)×100(mg/dl)

(2) TC (Total Cholesterol) in Blood

Test tubes for a blind test/standard/specimen were prepared, respectively. An enzyme solution of 3 ml was added to a blood specimen of 20 μl, and they were mixed well. They were reacted in a 37° C. water bath for 5 minutes. Using a blind test as a control, the absorbance of the standard and specimen was measured at a wavelength of 505 nm.

Total cholesterol (mg/dl)=(absorbance of specimen/absorbance of standard)×concentration of standard solution

(3) TG (Total Glyceride) in Blood

Test tubes for a blind test/standard/specimen were prepared, respectively. An enzyme solution of 3 ml was added to a blood specimen of 20 μl, and they were mixed well. They were reacted in a 37° C. water bath for 5 minutes. Using a blind test as a control, the absorbance of the standard and specimen was measured at a wavelength of 550 nm.

Amount of triglyceride (mg/dl)=(absorbance of specimen/absorbance of standard)×300(mg/dl)

(Amount of triglyceride of standard solution: 300 mg/dl)

2. Result

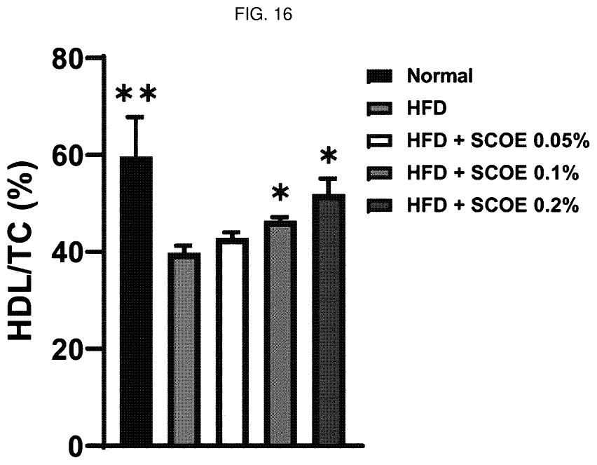

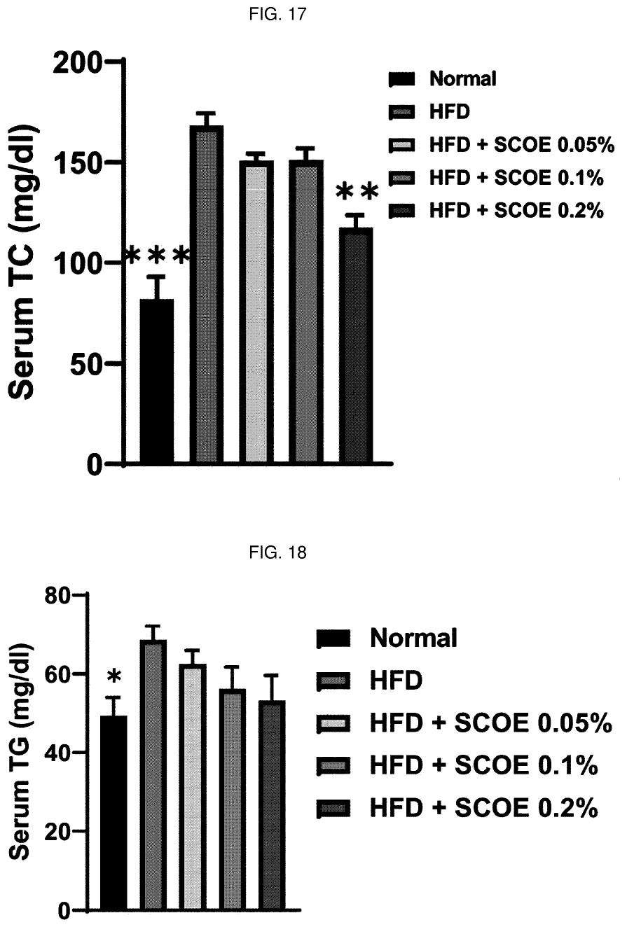

FIGS. 16 to 18 show the result of representing the HDL content compared to total cholesterol, the result of confirming the effect of reducing total cholesterol, and the result of confirming the effect of reducing triglyceride. Through these results, it was confirmed that the sea cucumber ovary extract treatment group showed an effect of reducing levels of cholesterol and triglyceride in blood. In particular, it should be noted here that although the total cholesterol numerical value was reduced, the HDL numerical value was increased. Through this result, it could be seen that it showed an effect for treatment of dyslipidemia.

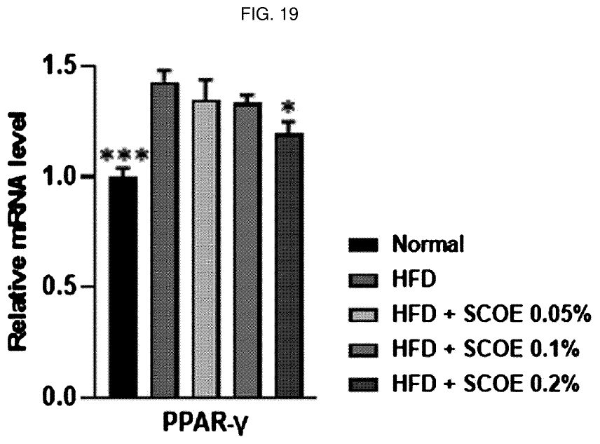

In addition, as a result of confirming expression of PPARγ known to be involved in inflammation regulation, in particular, inflammatory bowel disease and/or rheumatoid arthritis using the extract, an effect of reducing PPARγ compared to the high calorie diet could be confirmed. This result can be confirmed in FIG. 19. Through this, it was confirmed that the sea cucumber ovary extract could obtain the effect of inhibiting inflammation.

### INDUSTRIAL APPLICABILITY

The present invention can provide a composition for reducing body weight, a composition for inhibiting obesity, a composition for anti-diabetes, a composition for anti-inflammation, and a composition for improving dyslipidemia, which comprises a sea cucumber ovary extract. The composition of the present invention can provide a method for reducing body weight, a method for inhibiting obesity, a method for treating or improving diabetes, a method for treating or improving inflammation, and a method for treating or improving dyslipidemia.

1.-13. (canceled)

14. A composition comprising a therapeutically effective amount of a compound represented by the following chemical formula S1, S2, S3, S4 or S9 and a pharmaceutically acceptable carrier:

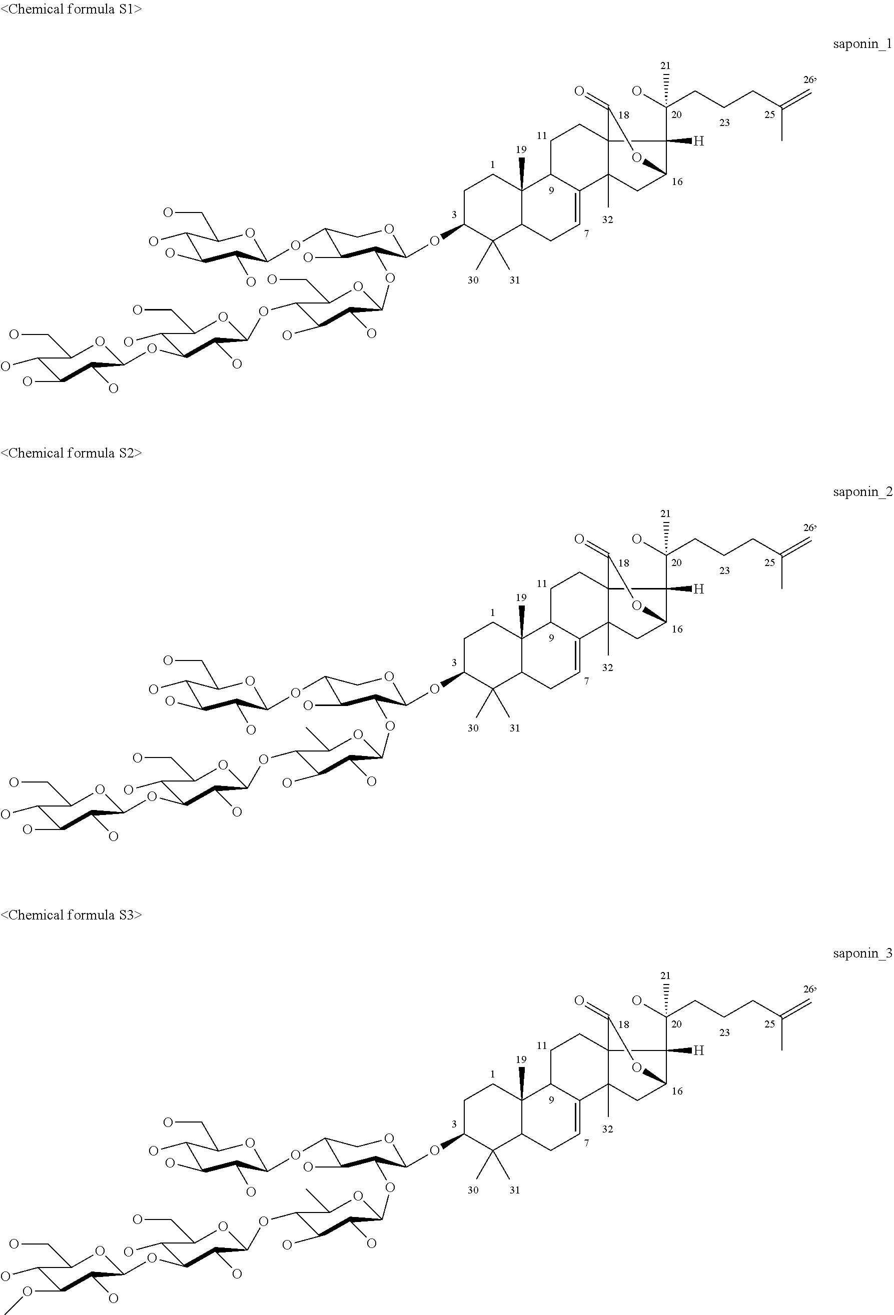

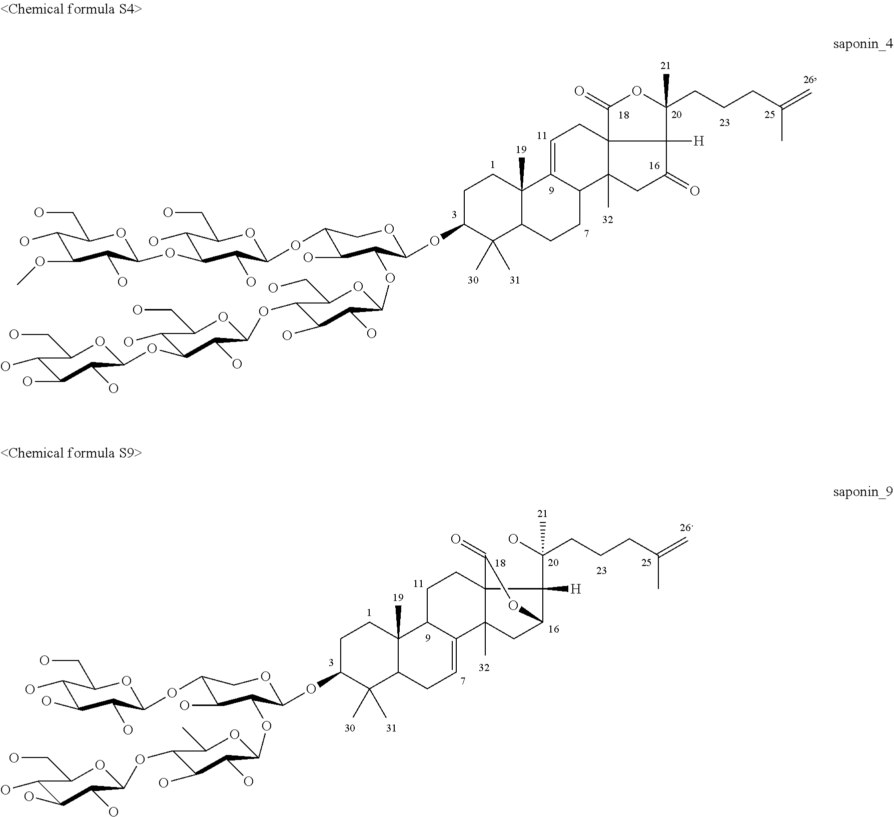

15. The composition according to claim 14, wherein the compound is extracted or separated from an ovary of a sea cucumber.

16. A method for treating or improving any one disease selected from the group consisting of obesity, inflammation, diabetes and dyslipidemia in a subject in need thereof, comprising:
administering a therapeutically effective amount of a sea cucumber genital gland extract, or a saponin compound derived from a sea cucumber genital gland to the subject.

17. The method according to claim 16, wherein the sea cucumber genital gland is an ovary of a sea cucumber.

18. The method according to claim 16, wherein the sea cucumber genital gland extract is obtained by extracting a sea cucumber genital gland with a 45 to 85% (V/V) ethanol aqueous solution as a solvent.

19. The method according to claim 18, wherein the sea cucumber genital gland extract is obtained by extracting a sea cucumber genital gland with a 48 to 55% (V/V) ethanol solvent.

20. The method according to claim 16, wherein the sea cucumber genital gland extract is a butanol fraction obtained by fractionizing an extract obtained by extracting a sea cucumber genital gland with a 48 to 55% (V/V) ethanol aqueous solution with butanol as a solvent.

21. The method according to claim 16, wherein the saponin compound comprises any one or more selected from the group consisting of the following chemical formulas S1, S2, S3, S4, S5, S6, S8 and S9:

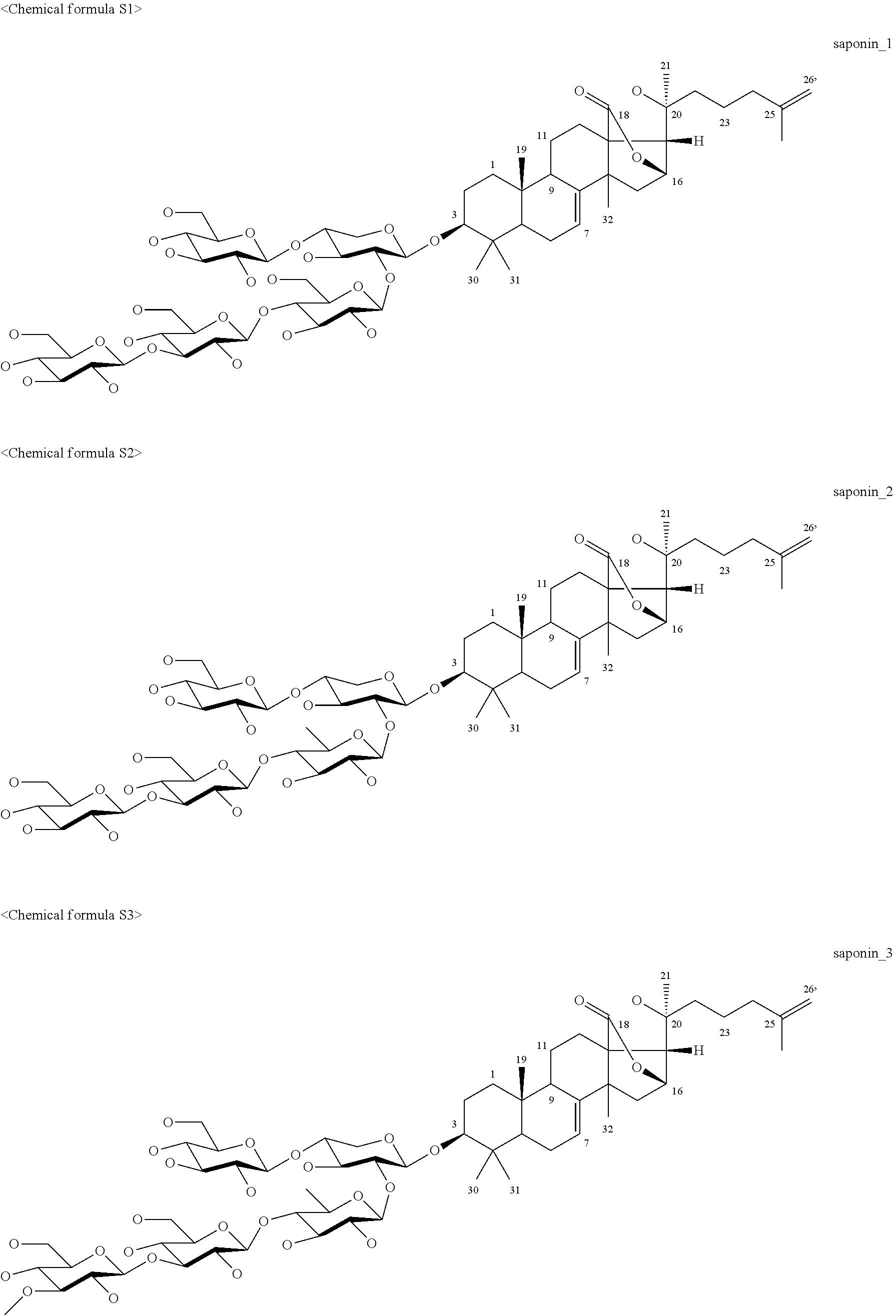

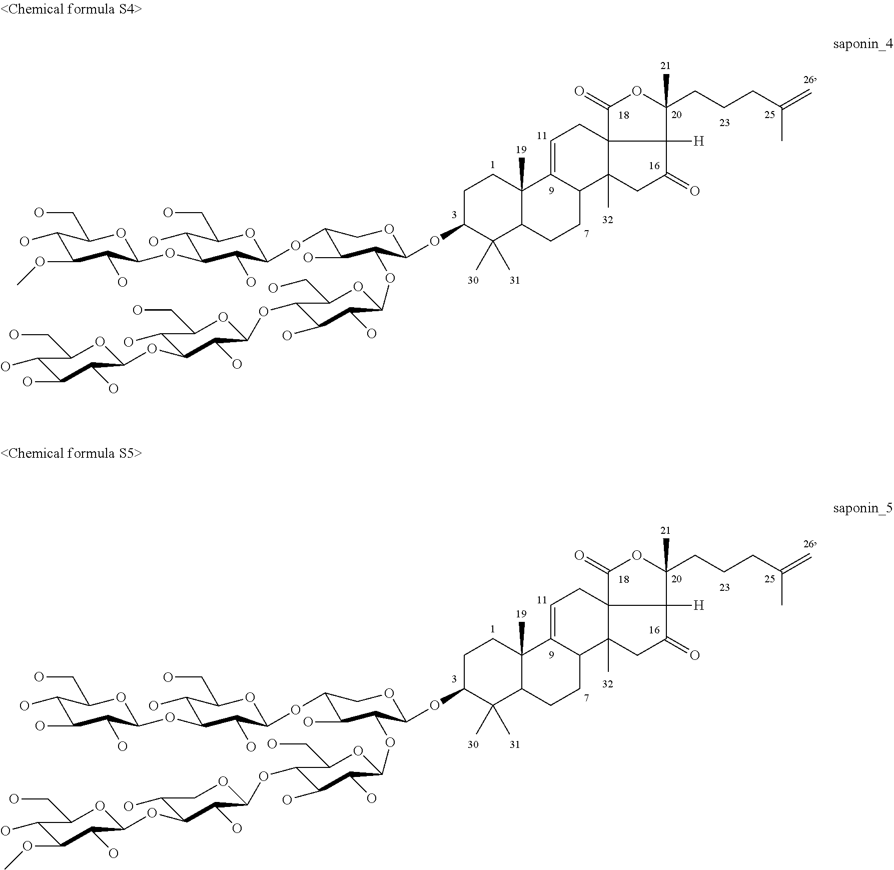

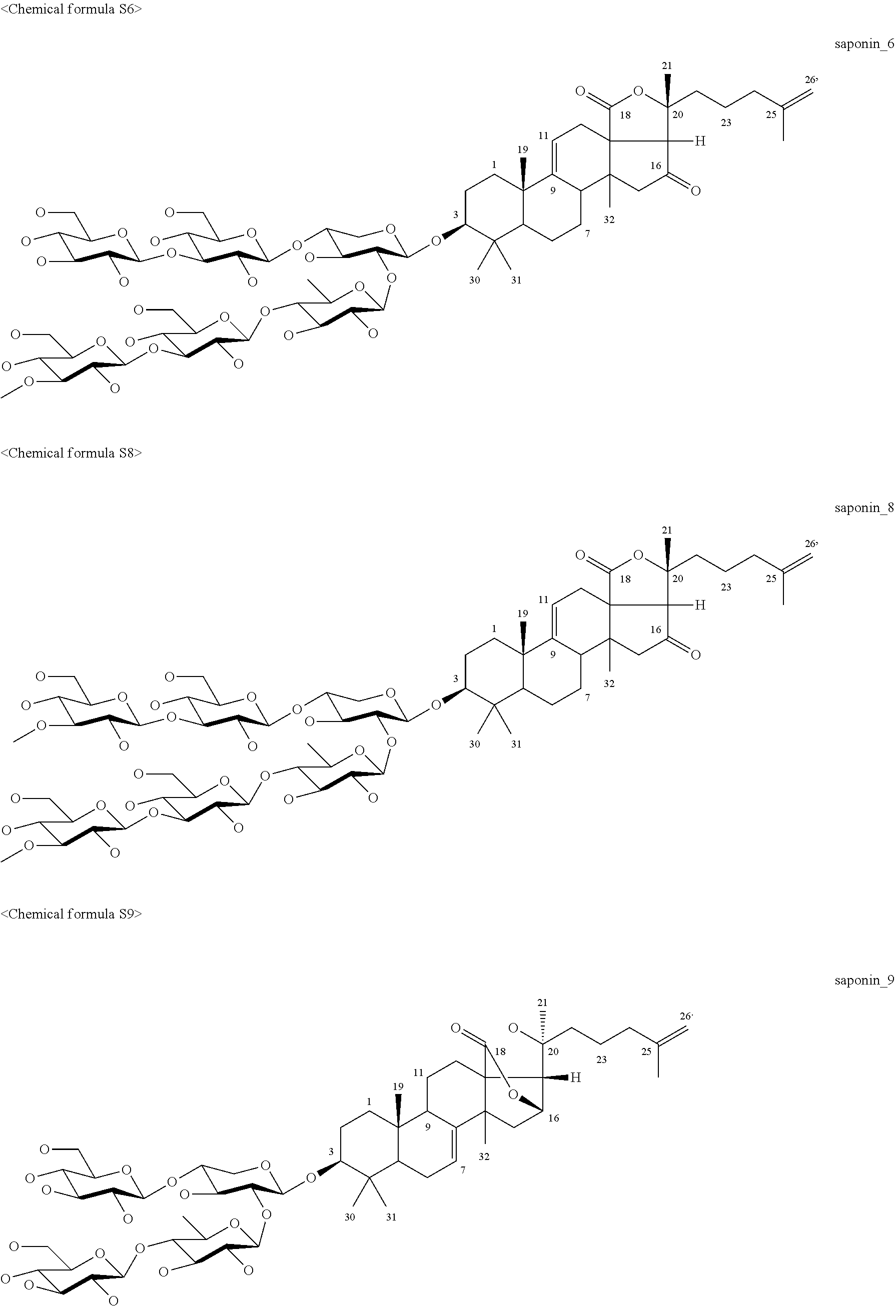

22. The method according to claim 16, wherein the sea cucumber genital gland extract, or saponin compound derived from the sea cucumber genital gland inhibits expression of any one or more proteins selected from the group consisting of PPARγ, FAS, aP2, and C/EBPα.

23. The method according to claim 16, wherein the sea cucumber genital gland extract, or saponin compound derived from the sea cucumber genital gland inhibits differentiation from a preadipocyte to an adipocyte, or inhibits intracellular fat accumulation.

24. A method for improving an effect of prevention or treatment of any one or more diseases selected from the group consisting of obesity, inflammation, diabetes and dyslipidemia of a sea cucumber genital gland extract, which increases any one or more saponin compounds selected from the group consisting of the following chemical formulas S1, S2, S3, S4, S5, S6, S8 and S9:

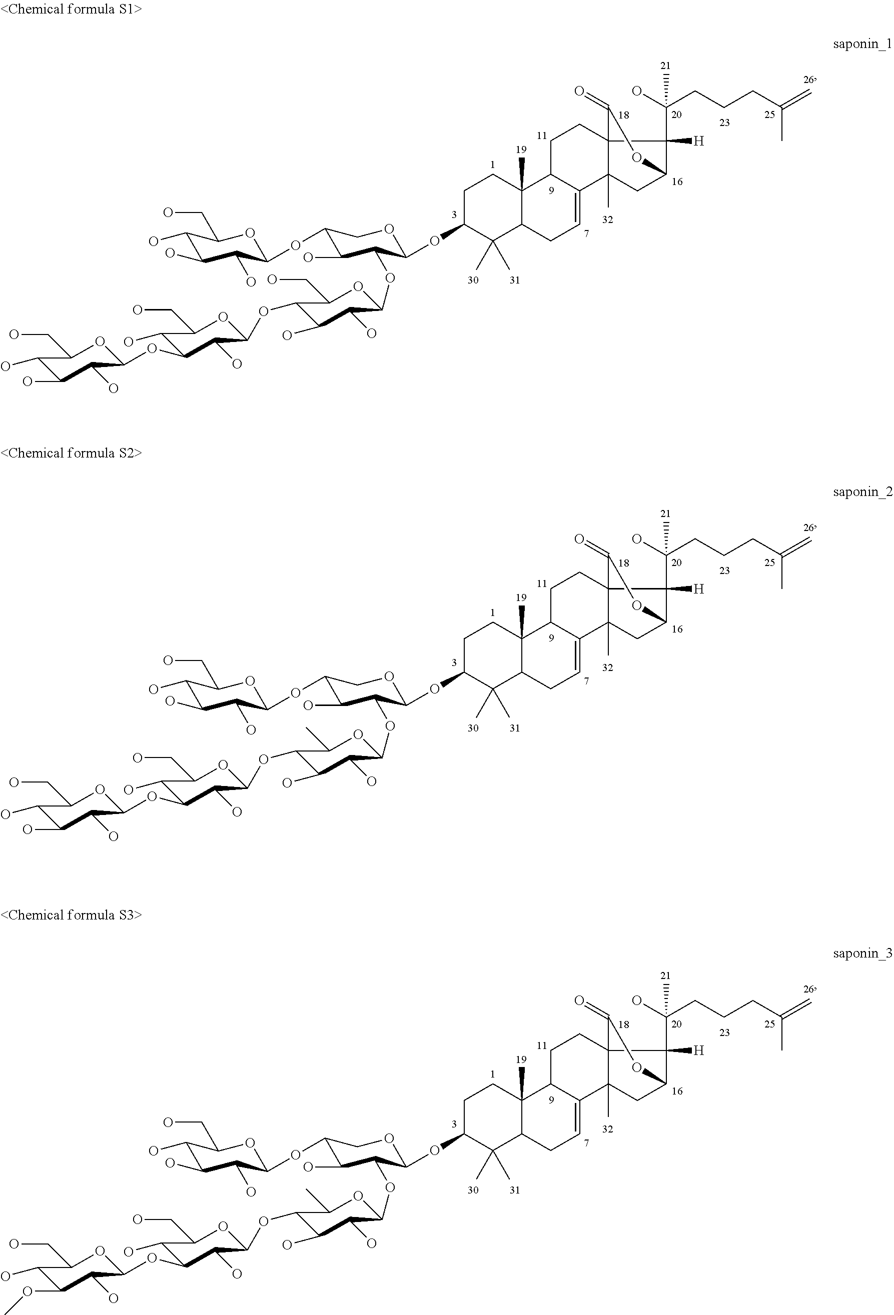

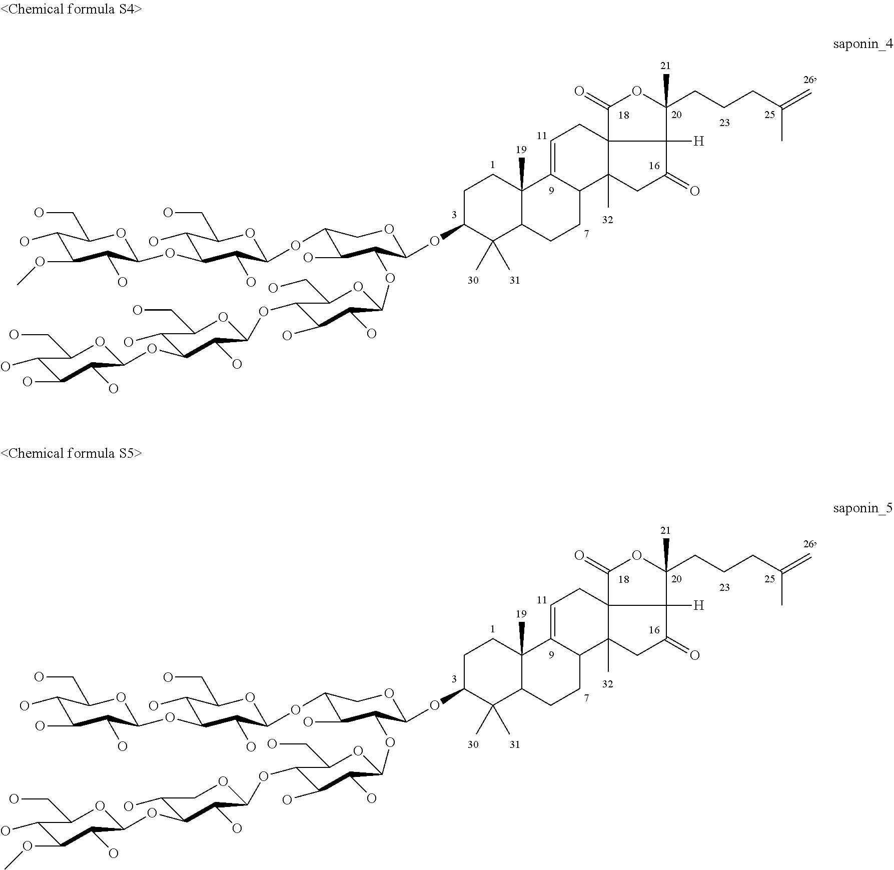

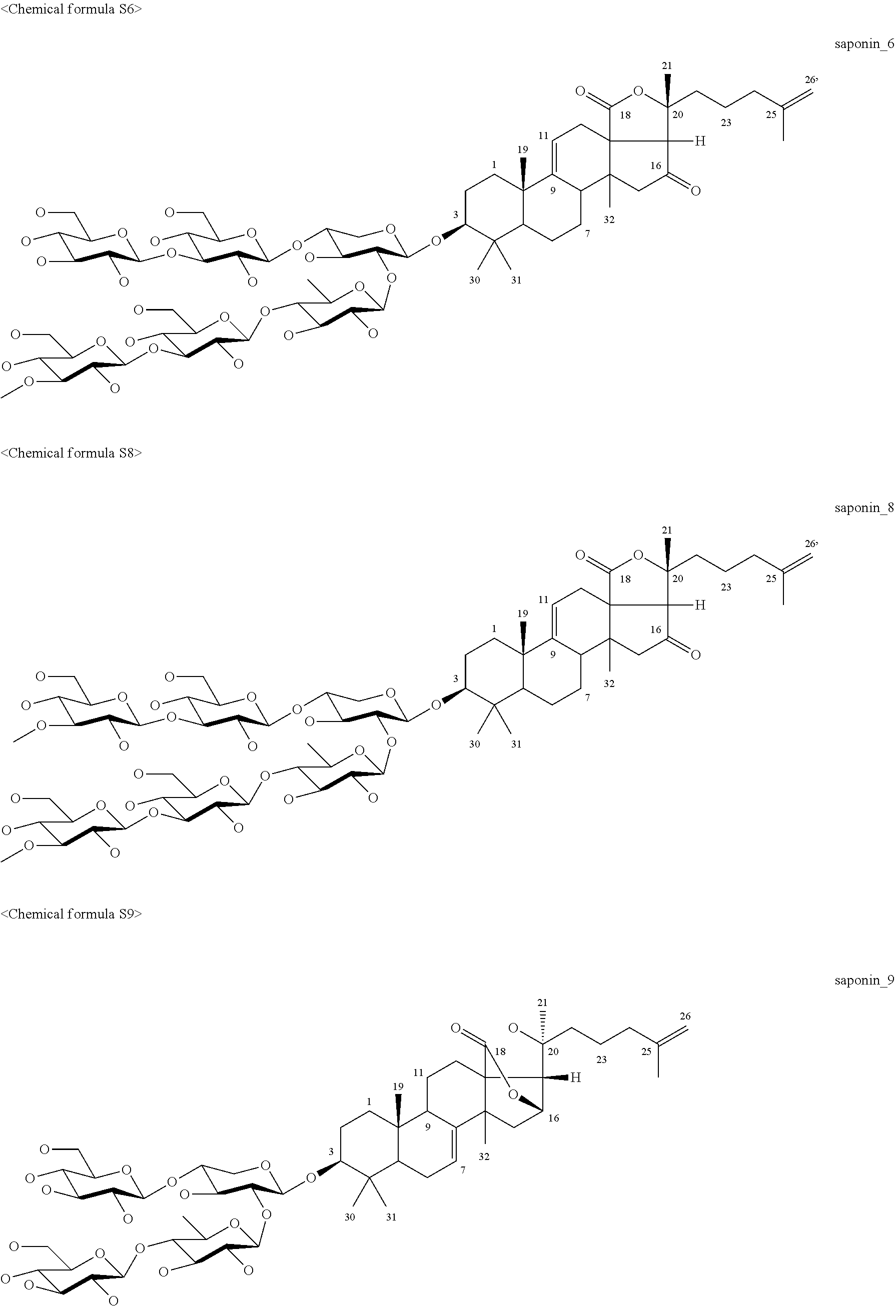

25. The method for improving an effect of prevention or treatment of any one or more diseases selected from the group consisting of obesity, inflammation, diabetes and dyslipidemia of a sea cucumber genital gland extract according to claim 24, wherein the sea cucumber genital gland extract is a sea cucumber ovary extract.

26. The method according to claim 24, wherein the method extracts a sea cucumber ovary with a 48 to 55% (V/V) ethanol aqueous solution, and obtains a butanol fraction from the extract.
